(19) United States
(12) Patent Application Publication
(10) Pub. No.: US 2003/0220556 A1
(43) Pub. Date: Nov. 27, 2003

(54) Method, system and device for tissue characterization

(75) Inventors: Yariv Porat, Haifa (IL); Itzhak Porat, Haifa (IL); Daniel Shach, Haifa (IL)

Correspondence Address: G. E. EHRLICH (1995) LTD., c/o ANTHONY CASTORINA, SUITE 207, 2001 JEFFERSON DAVIS HIGHWAY, ARLINGTON, VA, 22202, US

(73) Assignee: Vespro Ltd.

(21) Appl. No.: 10/435,749

(22) Filed: May 12, 2003

Related U.S. Application Data
(60) Provisional application No. 60/406,056, filed on Aug. 27, 2002.
(60) Provisional application No. 60/381,354, filed on May 20, 2002.

(51) Int. Cl. A61B 5/00

(52) U.S. Cl. 600/407; 601/2; 600/103; 601/84

(57) ABSTRACT

A method of characterizing a tissue present in a predetermined location of a body of a subject, the method comprising: generating mechanical vibrations at a position adjacent to the predetermined location, the mechanical vibrations are at a frequency ranging from 10 Hz to 10 kHz; scanning the frequency of the mechanical vibrations; and measuring a frequency response spectrum from the predetermined location, thereby characterizing the tissue.

This application claims the benefit of priority from U.S. Provisional Patent Application No. 60/406,056, filed Aug. 27, 2002 and U.S. Provisional Patent Application No. 60/381,354, filed May 20, 2002, the contents of which is hereby incorporated by reference.

### FIELD AND BACKGROUND OF THE INVENTION

The present invention relates to a medical system, method and device and, more particularly, to a medical system, method and apparatus particularly useful for tissue characterization. The present invention also relates to an endoscopic device which is useful for tissue characterization.

Medical technologies for examining the internal structure of tissues are of immense diagnostic importance. Internal body tissues are often examined to determine the structural details thereof and/or the flow of fluid therethrough in order to detect abnormalities, including pathologies, such as, but not limited to, cysts, tumors (benign and malignant), abscesses, mineral deposits, obstructions and anatomical defects.

One internal structural abnormally is atherosclerosis, which is an arterial disease in which fatty substances accumulate in the intima or inner media, the innermost membranes encompassing the lumen of the arteries. The resulting lesions are referred to as atherosclerotic plaques.

Clinical symptoms finally occur because the growing mass of the atherosclerotic plaque gradually constricts the inflicted artery and reduces blood flow therethrough, thereby compromising the function of a tissue or organ positioned downstream thereto.

Atherosclerosis and its complications, such as myocardial infarction, stroke and a variety of peripheral vascular diseases, such as gangrene of body extremes, remain major causes of morbidity and mortality in the modern world.

The plaques typically accumulate on the arterial wall in the form of pockets having a hard and flexible fibrous cover which does not easily crumble. This type of plaque is generally termed an “occlusive plaque”, and as long as it is stable and not overly constrictive, the inflicted subject is symptomatically undisturbed.

However, when the plaque pocket is covered with a soft, fatty wall, the wall tends to shed flakes downstream due to the fierce blood stream or due to flow associated cavitations. A flake migrating into the brain can cause Cerebro Vascular Accident (CVA). A flake migrating into the heart coronary system (CVD: Cardio Vascular Disease) can cause a stroke. A flake migrating into a leg via the femoral artery can, in the extreme case, cause gangrene. This type of plaque is therefore termed a “vulnerable plaque”. Statistics show that almost 80% of CVA and CVD deaths are due to vulnerable plaques rather than occlusive plaques, and therefore means with which to identify and cure vulnerable plaque are of a higher priority.

Left undetected, the formation of a plaque can result in the complete occlusion of the inflicted artery and lead to severe clinical consequences. For example, when complicated, the lesion becomes a calcified fibrous plaque, characterized by various degrees of necrosis, thrombosis and ulceration. With increasing necrosis and accumulation of cell debris, the arterial wall progressively weakens, and rupture of the intima can occur, causing aneurysm and hemorrhage. Arterial emboli can form when fragments of a plaque dislodge into the arterial lumen. Stenosis and impaired organ function result from gradual occlusion as plaques thicken and thrombi form.

Over the years, immersive attempts have been made both to detect and to identify internal structural abnormalities, with or without physically invading the body.

One such method is non-invasive ultrasound imaging. Ultrasonic images are formed by producing very short pulses of ultrasound using an electro-acoustic transducer, sending the pulses through the body, and measuring the properties (e.g., amplitude and phase) of the echoes from tissues within the body. Focused ultrasound pulses, referred to as “ultrasound beams”, are targeted to specific tissue regions-of-interest in the body. Typically, an ultrasound beam is focused at small lateral sections differing by predetermined depth intervals within the body to improve spatial resolution. Echoes are received by the ultrasound transducer and are processed to generate an image of the tissue or object in a region-of-interest. Ultrasonic imaging technology is presently used worldwide for examination of various internal structural abnormalities.

Another detection and classification method is the intravascular ultrasound (IVUS). Unlike in non-invasive ultrasound, in an IVUS system, an ultrasonic transducer is attached to an end of a catheter that is maneuvered through a patient's body to a point-of-interest such as within a blood vessel. The transducer is a single-element crystal or probe which is mechanically scanned or rotated back and forth to cover a sector over a selected angular range. Acoustic signals are transmitted during the scanning and echoes of these acoustic signals are received to provide data representative of the density of tissue over the sector. As the probe is swept through the sector, many acoustic lines are processed, building up a sector-shaped image of the patient. Once the data is collected, images of the blood vessel are reconstructed. A typical analysis includes determining the size of the lumen and amount and distribution of plaque in the analyzed vessel. The image data may show the extent of stenosis, reveal progression of disease, assist in determining whether procedures such as angioplasty or atherectomy are indicated or whether more invasive procedures may be advantageously warranted.

To date, many different types of IVUS measurements have been practiced. For example, imaging by IVUS “soft echo” using a compression ergonometer to determine the stiffness of a tissue was demonstrated in Hiro, T. et al., Am. Heart, J. 133(1) 1-7 (1997). Another work [de Corte et al., Circ. 8, 102(6) 617-23 (2000)] elaborated on IVUS sonoelasticity that yields a stiffness image of the arterial wall as the catheter moves forward. However, as these methods are mostly qualitative, the number of medical applications in which they can be used is limited. Moreover, the IVUS method is minimally invasive.

Another internal structural abnormality is a tumor, which may be malignant, and as such its eradication is promoted by early detection and treatment. One example of a malignant tumor is breast carcinoma, known as breast cancer.

The standard breast examination employed today in the detection of breast cancer is mammography, in which the breast is compressed between a source of x-rays and an x-ray sensitive film or plate, and x-rays are transmitted through the compressed breast tissue to expose the x-ray sensitive film or plate. The rays that pass through healthy tissue are moderately absorbed by the moderate density of the tissue, which causes healthy tissue to leave a gray shadow image on the x-ray sensitive film or plate. X-rays which pass through dense particles, such as calcifications characteristic of malignancy, undergo significant absorption, and the consequent deposit of relatively few photons on the x-ray film or plate leaves a bright spot thereon. X-rays which pass through very soft structures, such as cysts are only slightly absorbed, and leave a relatively dark spot on the x-ray sensitive film or plate.

Breast cancer can also be detected by ultrasound imaging in conjunction with mammography and/or hand-examination. Standard two dimensional ultrasound imaging has proven capable of detecting those calcified lesions which are also detectable by mammography. An example of the use of ultrasound imaging for detecting early calcification in breast is found in, for example, U.S. Pat. No. 5,997,477.

Once an abnormal tissue has been detected, it needs to be further diagnosed.

Although tissue biopsy is an extremely important diagnostic procedure for characterizing a tumor and for determining the most appropriate treatment for its eradication, the biopsy procedure can be preceded by non-invasive diagnostic techniques.

It is recognized herein that the desired diagnosis lies within the realm of the mechanical frequency response spectrum of the vibrating body tissue rather than in its shape, as yielded, e.g., by ultrasonic imaging. The reason for this recognition is that the mechanical characteristics of an examined tissue may be used to differentiate both between abnormal and normal tissues and between different types of abnormal tissues (e.g., benign or malignant tumors, different types of atherosclerotic plaques, etc.).

For Example, as is described hereinabove, blood vessel plaques are generally categorized into three major groups: (i) blood clots; (ii) occlusive plaques; and (iii) vulnerable plaques. These groups differ by the nature of their formation, their mechanical properties and the appropriate therapeutic treatment required once identified.

Thus, blood clots are soft and may present in many locations inside a blood vessel. Blood clots tend to sink on the arterial wall close to a bifurcation. The treatment for blood clots is by dissolving using specified enzymes.

Occlusive or fibrous plaque pocket wall may contain calcifications; hence it is heavier than normal intima tissue and sufficiently flexible to stay adhered to the arterial wall regardless of the blood flow. Nevertheless, the mechanical stiffness of fibrous plaque is higher than that of a normal blood wall.

Vulnerable or fatty plaque pocket wall is only slightly flexible and of a lower density than the normal arterial wall. Generally, a vulnerable plaque, which is considered to be the most dangerous plaque, does not follow the movement of the arterial wall and therefore may easily detach from the wall and migrate downstream with the blood flow.

Similarly, cysts, benign and malignant tumors have different mechanical properties, associated with their way of formation and constituents. Skin cancer of the Melanoma type appears as black, amorphous nevi. In their stage I and II development the nevi may not differ visually from other nevi or moles. Mechanically, nevertheless, malignant nevi are generally softer and lighter than healthy ones. In the case of breast cancer the cysts are not visually detectable, developing deep within the breast. These cists, or lesions, are usually harder and heavier than neighboring healthy tissue.

Hence, the mechanical properties of healthy and pathological tissues can be used as discriminators between different types of tissues and different types of pathologies, such as discriminating between an arterial wall and a plaque, discriminating between different types of plaques, discriminating between different types of tumors and healthy tissue, and the like.

Prior art teachings of the measurement of elastic moduli of tissues present in the body is based on ultrasound imaging of the mechanical properties of tissue, ultrasonic sonography, tactile sensor technology and other methods grouped under the definition of “sonoelasticity”.

The science of testing the elastic properties of living tissues is relatively young. Nevertheless, obtaining knowledge of the elastic and viscoelastic moduli of normal and abnormal (pathological) tissues is essential for the purpose of characterization and subsequent decision on the appropriate treatment.

A review of prior art attempts for tissue characterization is provided herein.

Early ex vivo experiments on canine aortic tissue [Yin FC, Circ. Res., 53(4) 464-72 (1983)] shed light on the measurement of the elastic modulus of fish via resonance. Parker K. J. et al. [Tissue Response to Mechanical Vibrations for “Sonoclasticity Imaging”, Ultrasound in Med. & Biol., vol. 16, No. 3, 1990, pp. 241-246] and Herrington [U.S. Pat. No. 6,264,609] used transverse excitation to measure the shear modulus in tissues. Measurement of tissue deformation under static pressure and comparison with simulations were carried out by Sarvazyan et al. [U.S. Pat. No. 5,524,636]. Recently, speed measurements of excited tissue were carried out by Sarvazyan et al. [U.S. Pat. No. 5,810,731].

Speed measurements, using a combined ultrasound—audio loudspeaker device operating in low frequency (10-1000 Hz) were performed by Lin et al. [U.S. Pat. Nos. 6,068,597 and 5,919,139] to obtain the elastic modulus of the tissue under excitation. The two acoustic effects were used simultaneously, transmission at one point and reception at another. However, the uneven attenuation of the ultrasonic signal was not compensated for in the low frequency resonance shape. A similar method was contemplated by Trahey et al. [U.S. Pat. No. 5,487,387] under the name ARFI (Acoustic Radiation Force Imaging). Another combination of sound and ultrasound was suggested by Fatemi et al. [Proc. Nat. Acad. Sci. 96, 6603-6608 (1999)]. The method practiced was to measure the reflections of bursts of ultrasound modulated by low frequency varying AM modulation.

Stiffness measurements of tissue were also made using the tactile sensors. P. M. P linkert et. al. (Bi-annual report 1 Jan. 1995-31 Dec. 1996, University of Tubingen, Germany) measured the dynamic force at the resonance of an impedance of freshly resected tissues. The research was motivated for providing tactile feedback during endoscopy by invasively touching an internal limb. The measurements showed difference in the resonance between internal benign and cancerous tissue. However, the modeling of this behavior is only partial, omitting dynamic parameters such force frequency response, or the resonance width.

Omata [U.S. Pat. No. 5,766,137] scanned the shift of a resonance frequency as a function of the mechanical load on the measured subject. In the method disclosed by Omata, a hardness measuring apparatus is first set to oscillate in a resonance state and then the operator initiates a contact between the apparatus and the subject's skin. Due to the impedance of the skin at the contact location, resonance frequency and voltage values are changed and monitored using appropriate measuring circuits. These changes, measured as a function of the load, are then used to determine the hardness of the tissue.

The frequency ranges used by Omata are of the order of 50 kHz, which frequency ranges result in several major drawbacks. First, a frequency of 50 KHz allows measuring resonance frequencies of the hardness measuring apparatus itself, as opposed to measuring the resonance frequencies of the tissues-of-interest. Second, as the typical resonance frequency of the tissues is of the order of few hundreds of Hz, the frequency changes which are to be measured are considerably small (of the order of 1%). Thus, some frequency changes may not be observed by the hardness measuring apparatus. Third, a skilled artisan would appreciate that a variation in the contact quality between the apparatus and the s kin result in a variation of the frequency and voltage reads. Given the low percentage the effect such variation may be crucial for determining the type of tissue. Forth, high frequency oscillations are known to allow measurements of tissues which are close to the contact location. Hence, for non-invasive procedures, only tissues which are close to the skin can be analyzed.

Another non-invasive method with which elastographic images are obtainable is Magnetic Resonance Imaging (MRI). Of interests are MRI scans that yield accurate elastographic images that show only qualitatively the nature of the various tissues in these scans [Van Huten E E et al. Magn. Reson. Med., 45(5) 827-37 (2001)]. Also, the price of such a procedure is substantially high

The present invention provides solutions to the problems associated with the prior art non-invasive techniques for tissue characterization.

### SUMMARY OF THE INVENTION

According to one aspect of the present invention there is provided a method of characterizing a tissue present in a predetermined location of a body of a subject, the method comprising: generating mechanical vibrations at a position adjacent to the predetermined location, the mechanical vibrations are at a frequency ranging from 10 Hz to 10 kHz; scanning the frequency of the mechanical vibrations; and measuring a frequency response spectrum from the predetermined location, thereby characterizing the tissue.

According to an additional aspect of the present invention there is provided a method of characterizing a tissue of a subject, the method comprising: (a) endoscopically inserting an endoscopic device into the subject, and using the endoscopic device for (i) imaging the subject so as to determine a position of the tissue; and (ii) generating mechanical vibrations at the position, the mechanical vibrations being at a frequency ranging from 10 Hz to 10 kHz; (b) scanning the frequency of the mechanical vibrations; and (c) measuring a frequency response spectrum from the tissue; thereby characterizing the tissue.

According to further features in preferred embodiments of the invention described below, the method further comprising measuring a phase angle as a function of the frequency.

According to still further features in the described preferred embodiments the method further comprising calculating at least one mechanical property of the tissue from the frequency response spectrum.

According to still further features in the described preferred embodiments the measuring the frequency response spectrum comprises measuring an amplitude as a function of the frequency.

According to still further features in the described preferred embodiments the step of generating mechanical vibrations is repeated a plurality of times, each time in a different location.

According to still further features in the described preferred embodiments the mechanical vibrations generating assembly comprises at least one mechanical linkage device for transferring the mechanical vibrations to the body.

According to another aspect of the present invention there is provided a system for characterizing a tissue present in a predetermined location of a body of a subject, the system comprising: a mechanical vibrations generating assembly for generating mechanical vibrations at a position adjacent to the predetermined location, the mechanical vibrations are at a frequency ranging from 10 Hz to 10 kHz; and a control unit for scanning the frequency of the mechanical vibrations, and for measuring a frequency response spectrum from the predetermined location, thereby to characterize the tissue.

According to further features in preferred embodiments of the invention described below, at least one of a size and a natural frequency of the mechanical linkage device is selected so as to increase dynamical interactions between the tissue and the at least one mechanical linkage device.

According to still further features in the described preferred embodiments the mechanical linkage device is characterized by a plurality of natural frequencies, where at least one frequency of the plurality of natural frequencies is higher than the frequency of the mechanical vibrations.

According to still further features in the described preferred embodiments the mechanical linkage device comprises a variable width beam spring.

According to still further features in the described preferred embodiments the mechanical linkage device comprises a strain gage for measuring displacement of the plurality of mechanical linkage devices.

According to still further features in the described preferred embodiments the mechanical linkage device comprises a proximity sensor for measuring displacement of the plurality of mechanical linkage devices.

According to still further features in the described preferred embodiments the system further comprising at least one additional mechanical vibrations generating assembly having a plurality of mechanical linkage devices being in mutual communication, and operable to generate mechanical vibrations at a position adjacent to the predetermined location

According to yet another aspect of the present invention there is provided an endoscopic device for in vivo characterization of a tissue of a subject, the device comprising: at least one imaging device for imaging the subject so as to determine a position of the tissue; and at least one mechanical vibrations generating assembly for generating mechanical vibrations at the position of the tissue, and for measuring a frequency response spectrum of the tissue, the mechanical vibrations are at a frequency ranging from 10 Hz to 10 kHz.

According to further features in preferred embodiments of the invention described below, the device further comprising a first mechanical linkage device connected to a first end of the tubular transducer and a second mechanical linkage device connected to a second end of the tubular transducer.

According to still further features in the described preferred embodiments the mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical device (MEMS) vibrating generating transducer assembly and an electrostatic mechanical vibrations generating transducer assembly.

According to still further features in the described preferred embodiments the mechanical vibrations generating assembly comprises a preamplifier, for at least partially amplifying electrical signals received from the at least one mechanical sensor.

According to still another aspect of the present invention there is provided a system for in vivo characterization of a tissue of a subject, the system comprising: an endoscopic device having at least one imaging device and at least one mechanical vibrations generating assembly, the at least one imaging device being for imaging the subject and the at least one mechanical vibrations generating assembly being for generating mechanical vibrations at a position of the tissue, the mechanical vibrations are at a frequency ranging from 10 Hz to 10 kHz; and a control unit for scanning the frequency of the mechanical vibrations, and for measuring a frequency response spectrum from the tissue, thereby to characterize the tissue.

According to further features in preferred embodiments of the invention described below, the mechanical vibrations generating assembly is operable to generate mechanical vibrations which are perpendicular to the tissue.

According to still further features in the described preferred embodiments the mechanical vibrations generating assembly is operable to generate mechanical vibrations which are inclined to the tissue by a predetermined inclination angle.

According to still further features in the described preferred embodiments the at least one mechanical linkage device comprises a first mechanical linkage device and a second mechanical linkage device.

According to still further features in the described preferred embodiments the system further comprising a first mechanical linkage device connected to a first end of the tubular transducer and a second mechanical linkage device connected to a second end of the tubular transducer.

According to still further features in the described preferred embodiments the imaging device is selected from the group consisting of an intra vascular ultra sound device, an intra vascular magnetic resonance device and a camera.

According to still further features in the described preferred embodiments the mechanical vibrations are perpendicular to the tissue.

According to still further features in the described preferred embodiments the mechanical vibrations are inclined to the tissue by a predetermined inclination angle.

According to still further features in the described preferred embodiments the mechanical vibrations generating assembly comprises at least one mechanical linkage device for transferring the mechanical vibrations to the tissue.

According to still further features in the described preferred embodiments at least one of a size and a natural frequency of the at least one mechanical linkage device is selected so as to increase dynamical interactions between the tissue and the at least one mechanical linkage device.

According to still further features in the described preferred embodiments the at least one mechanical linkage device comprises a variable width beam spring.

According to still further features in the described preferred embodiments the at least one mechanical linkage device comprises a strain gage for measuring displacement of the at least one mechanical linkage device.

According to still further features in the described preferred embodiments the at least one mechanical linkage device comprises a proximity sensor for measuring displacement of the at least one mechanical linkage device.

According to still further features in the described preferred embodiments the generating the mechanical vibrations is by transmitting mechanical vibration from a first mechanical linkage device to a second mechanical linkage device via at least one mechanical sensor.

According to still further features in the described preferred embodiments the method further comprising converting electrical signals into mechanical motions using a mechanical vibrations generating transducer assembly.

According to still further features in the described preferred embodiments the endoscopic device comprises an imaging device, selected from the group consisting of an intra vascular ultra sound device, an intra vascular magnetic resonance device and a camera.

According to still further features in the described preferred embodiments the method further comprising bulging the at least one contact-tip out of an encapsulation of the mechanical vibrations generating assembly so as to touch the tissue.

According to still further features in the described preferred embodiments the method further comprising at least partially amplifying electrical signals received from the at least one mechanical sensor.

According to still further features in the described preferred embodiments the transmitting the electrical comprises generating a synthesized electrical pulse.

According to still further features in the described preferred embodiments the method further comprising amplifying the synthesized electrical pulse.

According to still further features in the described preferred embodiments the method further comprising amplifying electrical signal transmitted from the mechanical vibrations generating assembly.

According to still further features in the described preferred embodiments the method further comprising displaying the electrical signal transmitted from the mechanical vibrations generating assembly.

According to still further features in the described preferred embodiments the method further comprising classifying the frequency response spectrum.

According to still further features in the described preferred embodiments the classifying the frequency response spectrum comprises: (a) identifying resonance peak maxima of the frequency response spectrum; (b) from the resonance peak maxima, determining a first type of maximum being indicative of a first type of tissue, and a second type of maximum being indicative of a second type of tissue; and (c) using the first type of maximum and the second type of maximum to classify the first and the types of tissue.

According to still further features in the described preferred embodiments the step (c) comprises calculating a ratio between the first type of maximum and the second type of maximum.

According to still further features in the described preferred embodiments the method further comprising averaging the resonance peak maxima.

According to still further features in the described preferred embodiments the first and the second types of maxima are determined by absolute values of the resonance peak maxima.

According to still further features in the described preferred embodiments the first and the second types of maxima are determined by shapes of the resonance peak maxima.

According to still further features in the described preferred embodiments the first and the second types of maxima are determined by frequency shifts of the resonance peak maxima.

According to still further features in the described preferred embodiments the classifying comprises: (a) constructing a physical model of a plurality of harmonic oscillators, the physical model comprises a set of parameters and being characterized by a plurality of equations of motion; (b) simultaneously solving the plurality of equations of motion so as to provide at least one frequency response; and (c) comparing the at least one frequency response with the frequency response spectrum; thereby classifying the frequency response spectrum.

According to still further features in the described preferred embodiments the method further comprising repeating the steps (a)-(c) at least once, each time using different set of parameters.

According to yet an additional aspect of the present invention there is provided a system for characterizing a tissue present in a predetermined location of a body of a subject, the system comprising: at least one mechanical vibrations generating assembly each having a plurality of mechanical linkage devices being in mutual communication, and operable to generate mechanical vibrations at a position adjacent to the predetermined location, the mechanical vibrations being at a frequency ranging from 10 Hz to 10 kHz; and a control unit for scanning the frequency of the mechanical vibrations, and for measuring a frequency response spectrum from the tissue, thereby to characterize the tissue.

According to further features in preferred embodiments of the invention described below, at least one of a size and a natural frequency of the plurality of mechanical linkage devices is selected so as to increase dynamical interactions between the tissue and the at least one mechanical linkage device.

According to still further features in the described preferred embodiments the at least one mechanical linkage device is characterized by a plurality of natural frequencies, and further wherein at least one frequency of the plurality of natural frequencies is higher than the frequency of the mechanical vibrations.

According to still further features in the described preferred embodiments the plurality of mechanical linkage devices comprises a variable width beam spring.

According to still further features in the described preferred embodiments the plurality of mechanical linkage devices comprises a strain gage for measuring displacement of the plurality of mechanical linkage devices.

According to still further features in the described preferred embodiments the plurality of mechanical linkage devices comprises a proximity sensor for measuring displacement of the plurality of mechanical linkage devices.

According to still further features in the described preferred embodiments the control unit is operable to measure an amplitude as a function of the frequency.

According to still further features in the described preferred embodiments the control unit is operable to measure a phase angle as a function of the frequency.

According to still further features in the described preferred embodiments the control unit is operable to calculate at least one mechanical property of the tissue from the frequency response spectrum.

According to still further features in the described preferred embodiments the mechanical property is an elastic constant.

According to still further features in the described preferred embodiments the mechanical property is selected from the group consisting of an elastic modulus, a Poisson's ratio, a shear modulus, a bulk modulus and a first Lamé coefficient.

According to still further features in the described preferred embodiments the position is on a skin of the body.

According to still further features in the described preferred embodiments the position is close to a blood vessel-of-interest.

According to still further features in the described preferred embodiments the blood vessel-of-interest is selected from the group consisting of a carotid, a femoral vessel and an abdominal aorta.

According to still further features in the described preferred embodiments the position is close to a lesion selected from the group consisting of a dermal lesion, a sub-dermal lesion and an internal lesion.

According to still further features in the described preferred embodiments the position is close to a bone.

According to still further features in the described preferred embodiments the position is close to a thorax.

According to still further features in the described preferred embodiments the mechanical vibrations generating assembly is operable to generate mechanical vibrations which are perpendicular to the body.

According to still further features in the described preferred embodiments the mechanical vibrations generating assembly is operable to generate mechanical vibrations which are inclined to the body by a predetermined inclination angle.

According to still further features in the described preferred embodiments the plurality of mechanical linkage devices comprises a first mechanical linkage device and a second mechanical linkage device.

According to still further features in the described preferred embodiments the first and the second mechanical linkage devices are connected by at least one mechanical sensor, capable of receiving mechanical vibration therebetween.

According to still further features in the described preferred embodiments the first and the second mechanical linkage devices are connected by at least one connection rod.

According to still further features in the described preferred embodiments the mechanical vibrations generating transducer assembly comprises a tubular transducer.

According to still further features in the described preferred embodiments the plurality of mechanical linkage devices comprises a first mechanical linkage device connected to a first end of the tubular transducer and a second mechanical linkage device connected to a second end of the tubular transducer.

According to still further features in the described preferred embodiments the mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly and an electrostatic mechanical vibrations generating transducer assembly.

According to still further features in the described preferred embodiments the mechanical vibrations generating assembly is sizewise compatible with an anatomical system selected from the group consisting of the vascular system, the cardio-vascular system and the urinary system.

According to still further features in the described preferred embodiments the system further comprising an imaging device for imaging the tissue.

According to still further features in the described preferred embodiments the imaging device is selected from the group consisting of an intra vascular ultra sound device, an intra vascular magnetic resonance device, a camera, a computer tomography device, and a magnetic resonance device.

According to still further features in the described preferred embodiments the imaging device is in communication with the control unit.

According to still further features in the described preferred embodiments the communication is selected from the group consisting of optical communication, electrical communication and acoustical communication.

According to still further features in the described preferred embodiments the imaging device is connected to the mechanical vibrations generating assembly.

According to still further features in the described preferred embodiments the mechanical vibrations generating assembly comprises a posing mechanism for bulging the at least one contact-tip out of an encapsulation of the mechanical vibrations generating assembly so as to touch the tissue.

According to still further features in the described preferred embodiments the mechanical vibrations generating assembly comprises a preamplifier, for at least partially amplifying electrical signals received from the at least one mechanical sensor.

According to still further features in the described preferred embodiments the control unit comprises a transmission unit for transmitting an electrical signal to the mechanical vibrations generating assembly.

According to still further features in the described preferred embodiments the transmission unit comprises a computerized synthesizer for generating a synthesized electrical pulse.

According to still further features in the described preferred embodiments the transmission unit further comprises a power amplifier for amplifying the synthesized electrical pulse.

According to still further features in the described preferred embodiments the control unit comprises a receiver for receiving an electrical signal from the mechanical vibrations generating assembly.

According to still further features in the described preferred embodiments the receiver comprises a preamplifier and a line amplifier, the preamplifier and the line amplifier configured and designed to amplify the electrical signal transmitted from the mechanical vibrations generating assembly.

According to still further features in the described preferred embodiments the receiver further comprises a display for displaying the electrical signal transmitted from the mechanical vibrations generating assembly.

According to still an additional aspect of the present invention there is provided a mechanical vibrations generating assembly for generating mechanical vibrations at a position of a body of a subject, comprising a transducer assembly, a first mechanical linkage device, connected to a first end of the transducer assembly, and a second mechanical linkage device, connected to a second end of the transducer assembly; wherein the transducer assembly, the first mechanical linkage device and the second mechanical linkage device are constructed and designed so that when electrical signals are inputted to the transducer assembly, the electrical signals are converted into mechanical motions, and the first and the second mechanical linkage devices generates the mechanical vibrations.

According to further features in preferred embodiments of the invention described below, the mechanical vibrations generating assembly further comprising at least one additional mechanical linkage device, mechanically communicating with the transducer assembly.

According to still further features in the described preferred embodiments the first and the second mechanical linkage devices are each independently membranes.

According to still further features in the described preferred embodiments the membranes are made of a material selected from the group consisting of a plastic and a metal.

According to still further features in the described preferred embodiments the membranes are piezo-polymeric membranes.

According to still further features in the described preferred embodiments the mechanical vibrations generating assembly further comprising at least one contact-tip, connected to at least one of the mechanical linkage devices.

According to still further features in the described preferred embodiments at least one of a size and a natural frequency of the mechanical linkage devices is selected so as to increase dynamical interactions between the a portion of the body and the mechanical linkage devices.

According to still further features in the described preferred embodiments the mechanical linkage devices are characterized by a plurality of natural frequencies, where at least one frequency of the plurality of natural frequencies is higher than a frequency of the mechanical vibrations.

According to still further features in the described preferred embodiments the plurality of mechanical linkage devices comprises a strain gage for measuring displacement of the mechanical linkage devices.

According to still further features in the described preferred embodiments the mechanical linkage devices comprises a proximity sensor for measuring displacement of the mechanical linkage devices.

According to still further features in the described preferred embodiments the transducer assembly comprises a tubular transducer.

According to still further features in the described preferred embodiments the mechanical vibrations generating assembly further comprising at least one mechanical sensor.

According to still further features in the described preferred embodiments the mechanical vibrations generating assembly further comprising at least one mechanical sensor connecting the first mechanical linkage device and the mechanical linkage device, the at least one mechanical sensor being capable of receiving mechanical vibration therethrough.

According to a further aspect of the present invention there is provided a method of classifying a frequency response spectrum of a structural material, the method is executable by a data processor and comprising; (a) constructing a physical model of a plurality of harmonic oscillators, the physical model comprises a set of parameters and being characterized by a plurality of equations of motion; (b) simultaneously solving the plurality of equations of motion so as to provide at least one frequency response; and (c) comparing the at least one frequency response with the frequency response spectrum of the structural material, thereby classifying the frequency response spectrum of the structural material.

According to further features in preferred embodiments of the invention described below, the method further comprises repeating the steps (a)-(c) at least once, each time using a different set of parameters.

According to yet a further aspect of the present invention there is provided an apparatus for classifying a frequency response spectrum of a structural material, the apparatus comprising; (a) a constructor for constructing a physical model of a plurality of harmonic oscillators, the physical model comprises a set of parameters and being characterized by a plurality of equations of motion; (b) a solver for simultaneously solving the plurality of equations of motion so as to provide at least one frequency response; and (c) a comparing unit for comparing the at least one frequency response with the frequency response spectrum of the structural material, thereby to classify the frequency response spectrum of the structural material.

According to further features in preferred embodiments of the invention described below, the physical model is an N degree-of-freedom physical model, the N is a positive integer.

According to still further features in the described preferred embodiments the plurality of harmonic oscillators are coupled harmonic oscillators.

According to still further features in the described preferred embodiments at least a portion of the plurality of harmonic oscillators are damped harmonic oscillators.

According to still further features in the described preferred embodiments at least a portion of the plurality of harmonic oscillators are forced harmonic oscillators.

According to still further features in the described preferred embodiments the set of parameters comprises at least one constant of inertia and at least one elastic constant.

According to still further features in the described preferred embodiments the constant of inertia is mass and the elastic constant is a spring constant.

According to still further features in the described preferred embodiments the constant of inertia is inductance and the elastic constant is a reciprocal of capacitance.

According to still further features in the described preferred embodiments the set of parameters represent dynamic stiffness and density of the structural material.

According to still a further aspect of the present invention there is provided a method of constructing a frequency resonance spectra library the frequency resonance spectra characterizing a plurality of tissues of a plurality of subjects, the method comprising, for each subject: (a) selecting a tissue of the subject and generating mechanical vibrations at a position adjacent to the tissue, the mechanical vibrations are at a frequency ranging from 10 Hz to 10 kHz; (b) scanning the frequency of the mechanical vibrations; (c) measuring a frequency response spectrum from of the tissue; and (d) recording the frequency response spectrum; thereby providing a frequency response spectrum entry of the library, the frequency response spectrum entry characterizing the tissue, thereby constructing the frequency resonance spectra library.

According to further features in preferred embodiments of the invention described below, the mechanical vibrations are perpendicular to the body.

According to still further features in the described preferred embodiments the generating the mechanical vibrations is performed such that the mechanical vibrations are inclined to the body, by a predetermined inclination angle.

According to still further features in the described preferred embodiments the predetermined inclination angle is selected so as to enhance data acquisition.

According to still further features in the described preferred embodiments the step of generating mechanical vibrations is repeated a plurality of times, each time with a different inclination angle.

According to still further features in the described preferred embodiments the step of generating mechanical vibrations is repeated a plurality of times, each time for a different tissue.

According to still further features in the described preferred embodiments the frequency of the mechanical vibrations is selected from the group consisting of a single frequency, a superposition of a plurality of frequencies, a continuous frequency scan (chirp), and a band-limited white noise frequency.

According to still further features in the described preferred embodiments the generating the mechanical vibrations is by a mechanical vibrations generating assembly.

According to still further features in the described preferred embodiments the mechanical vibrations generating assembly is constructed and designed so as to minimize effects of environmental noise.

According to still further features in the described preferred embodiments the mechanical vibrations generating assembly comprises a mechanical linkage device for transferring the mechanical vibrations to the body.

According to still further features in the described preferred embodiments the mechanical vibrations generating assembly comprises at least one contact-tip.

According to still further features in the described preferred embodiments the at least one contact-tip comprises a plurality of contact-tips arranged in a matrix-like arrangement.

According to still further features in the described preferred embodiments the at least one contact-tip is sterilizable.

According to still further features in the described preferred embodiments the at least one contact-tip comprises at least one sterilizable cover.

According to still further features in the described preferred embodiments the at least one contact-tip is disposable.

According to still further features in the described preferred embodiments the mechanical vibrations generating assembly comprises a mechanical vibrations generating transducer assembly, the mechanical vibrations generating transducer assembly is operable to convert electrical signals into mechanical motions.

According to still further features in the described preferred embodiments the mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly, and an electrostatic mechanical vibrations generating transducer assembly.

According to still further features in the described preferred embodiments the mechanical vibrations generating assembly comprises at least one mechanical sensor.

According to still further features in the described preferred embodiments the at least one mechanical sensor is selected from the group consisting of a contact sensor and a remote sensor.

According to still further features in the described preferred embodiments the at least one mechanical sensor is selected from the group consisting of an acceleration sensor, a force sensor, a pressure sensor and a displacement sensor.

According to still further features in the described preferred embodiments the mechanical vibrations generating assembly comprises a mechanism for isolating the mechanical vibrations generating assembly from environmental vibrations.

According to still further features in the described preferred embodiments the mechanism is operable to independently move in three orthogonal directions.

According to still further features in the described preferred embodiments the mechanism is operable to independently rotate in at least two orthogonal directions.

According to still further features in the described preferred embodiments the method further comprising transmitting an electrical signal to the mechanical vibrations generating assembly.

According to still further features in the described preferred embodiments the measuring is by receiving an electrical signal transmitted from the mechanical vibrations generating assembly.

According to still further features in the described preferred embodiments the method further comprising displaying the electrical signal transmitted from the mechanical vibrations generating assembly on a display.

According to still further features in the described preferred embodiments the display is selected from the group consisting of an oscilloscope, a spectrum analyzer, a processor display and a printer.

According to still a further aspect of the present invention there is provided a resonance spectra library produced by at least one of the methods of the present invention, the resonance spectra of the library are stored, in a retrievable and/or displayable format, on a memory media.

According to still a further aspect of the present invention there is provided a memory media, storing in a retrievable and/or displayable format the resonance spectra of the resonance spectra library.

According to further features in preferred embodiments of the invention described below, the tissue forms a part of, or is associated with, the urinary system of the subject.

According to still further features in the described preferred embodiments the tissue forms a part of an organ.

According to still further features in the described preferred embodiments the tissue forms a part of an internal organ.

According to still further features in the described preferred embodiments the tissue forms a portion of a tumor.

According to still further features in the described preferred embodiments the tissue forms a portion of an internal tumor.

According to still further features in the described preferred embodiments the tissue is a pathological tissue.

According to still further features in the described preferred embodiments the tissue forms a part of, or is associated with, a blood vessel tissue.

According to still further features in the described preferred embodiments the blood vessel tissue is selected from the group consisting of a blood clot, an occlusive plaque and a vulnerable plaque.

According to still further features in the described preferred embodiments the blood vessel is selected from the group consisting of a carotid, a femoral, and an abdominal aorta.

According to still further features in the described preferred embodiments the tissue forms a portion of a bone.

According to still further features in the described preferred embodiments the tissue is a stenotic tissue.

According to still further features in the described preferred embodiments the tissue is a lesion.

According to still further features in the described preferred embodiments the lesion is selected from the group consisting of a dermal lesion, a sub-dermal lesion and an internal lesion.

According to still further features in the described preferred embodiments the position is close to an internal lesion.

According to still further features in the described preferred embodiments the adjacent to the tissue is on a skin of the body.

The present invention successfully addresses the shortcomings of the presently known configurations by providing a method, system and device for characterizing a tissue present in a body of a subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a processor using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a system for characterizing a tissue, which comprises a mechanical vibrations generating assembly and a control unit, according to the present invention;

FIG. 2a illustrates a typical configuration of the mechanical vibrations generating assembly, according to the present invention;

FIG. 2b illustrates a cross sectional view of the mechanical vibrations generating assembly, in the embodiment in which more than one mechanical linkage device is used, according to the present invention;

FIG. 2c illustrates an endoscopic device for in vivo characterization of a tissue, according to the present invention;

FIG. 3 illustrates the control unit which comprises a transmission unit, a receiver and a processor, according to the present invention;

FIG. 4 is a system of a plurality of degrees-of-freedom each degree-of-freedom is constrained to a one dimensional motion, according to the present invention;

FIG. 5 shows a normalized amplitude as a function of a normalized frequency, for an excitation of one dimensional systems, representing added hard plaque and benign artery, according to the present invention;

FIG. 6 shows a phase angle as a function of a normalized frequency, for an excitation of one dimensional systems, representing added hard plaque and benign artery, according to the present invention;

FIG. 7 shows a normalized amplitude as a function of a normalized frequency, for low normalized frequency excitation of one dimensional systems, representing added hard plaque and benign artery, according to the present invention;

FIG. 8 shows phase angle as a function of a normalized frequency, for low normalized frequency excitation of one dimensional systems, representing added hard plaque and benign artery, according to the present invention;

FIG. 9 shows a normalized amplitude as a function of a normalized frequency, for excitation of one dimensional systems, representing benign arterial tissue and stiffened arterial tissue, according to the present invention;

FIG. 10 shows a phase angle as a function of a normalized frequency, for excitation of one dimensional systems, representing benign arterial tissue and stiffened arterial tissue, according to the present invention;

FIG. 11 shows a normalized amplitude as a function of a normalized frequency, for low normalized frequency excitation of one dimensional systems, representing benign arterial tissue and stiffened arterial tissue, according to the present invention;

FIG. 12 shows a phase angle as a function of a normalized frequency, for low normalized frequency excitation of one dimensional systems, representing benign arterial tissue and stiffened arterial tissue, according to the present invention;

FIG. 13 illustrates an artery carrying a plaque, which is located on the wall of the artery, according to the present invention;

FIG. 14a illustrates a two dimensional model which consists of a plurality of particles, according to the present invention;

FIG. 14b illustrates coupling of a certain particle of the two dimensional model with its eight neighbours, according to the present invention;

FIG. 14c illustrates forces, spring, and viscous damper between two neighboring particles of the two dimensional model, according to the present invention;

FIG. 14d shows a square region of particles of the two dimensional model, which simulates an artery, according to the present invention;

FIG. 15 shows a normalized amplitude, as a function of the normalized frequency for excitation in x direction of two dimensional models representing hard plaque and soft plaque, according to the present invention;

FIG. 16 shows a phase angle, as a function of the normalized frequency for excitation in x direction of two dimensional models representing hard plaque and soft plaque, according to the present invention;

FIG. 17 shows a normalized amplitude, as a function of the normalized frequency for excitation in y direction of two dimensional models representing hard plaque and soft plaque, according to the present invention;

FIG. 18 shows a phase angle, as a function of the normalized frequency for excitation in y direction of two dimensional models representing hard plaque and soft plaque, according to the present invention;

FIG. 19 shows a normalized amplitude, as a function of the normalized frequency for excitation in x direction of two dimensional models representing hard plaque and benign clean artery, according to the present invention;

FIG. 20 shows a phase angle, as a function of the normalized frequency for excitation in x direction of two dimensional models representing hard plaque and benign clean artery, according to the present invention;

FIG. 21 shows a normalized amplitude, as a function of the normalized frequency for center and side excitations in x direction of two dimensional models representing hard plaque, according to the present invention;

FIG. 22 shows a phase angle, as a function of the normalized frequency for center and side excitations in x direction of two dimensional models representing hard plaque, according to the present invention;

FIG. 23 illustrates a model representing a suspected region of a skin having a benign region and the lesion, according to the present invention.

FIG. 24 shows a normalized amplitude, as a function of the normalized frequency for excitation in the x direction for excitation of benign skin tissue and malignant lesion in x direction, according to the present invention;

FIG. 25 shows a phase angle, as a function of the normalized frequency for excitation in the x direction for excitation of benign skin tissue and malignant lesion in x direction, according to the present invention.

FIGS. 26a-c schematically exemplify a mechanical linkage device, according to a preferred embodiment of the present invention;

FIG. 27 shows an experimental setup for simulating a tissue;

FIG. 28 shows the absolute value and the phase of the frequency response as a function of the frequency, as measured using the experimental setup.

FIG. 29 is a three-dimensional plot of the response acquired from a copper insert, using the experimental setup;

FIG. 30 is a three-dimensional plot of the response acquired from a rubber insert, using the experimental setup;

FIG. 31 shows a projection of FIG. 29 on the frequency-amplitude plane;

FIG. 32 shows a projection of FIG. 29 on the distance-amplitude plane;

FIG. 33 shows a projection of FIG. 30 on the frequency-amplitude plane;

FIG. 34 shows a projection of FIG. 30 on the distance-amplitude plane;

FIG. 35 shows the lower resonance frequency shift for a copper insert;

FIG. 36 shows the upper resonance frequency shift for a copper insert;

FIG. 37 shows the absolute value of the frequency response function obtained for copper insert at the lower resonance frequency; and

FIG. 38 shows the absolute value of the frequency response function obtained for copper insert at the upper resonance frequency

### DESCRIPTION OF THE PREFERRED EMBODIMENTS

The present invention is of a method, system and device for characterizing a tissue present in a predetermined location of a body of a subject, which can be used for non-invasive and minimal-invasive (e.g., catheter based) medical diagnostics. More particularly, the method, system and device of the present invention can be used for classifying the frequency response spectrum of tissue structures within the body, to thereby provide for non-invasive or minimal invasive medical diagnostics. Specifically, the present invention can be used to characterize and identify a variety of tissues and pathologies in the body, such as, but not limited to, plaques, lesions, tumors, cysts and the like.

The principles and operation of a method, apparatus and system for characterizing a tissue according to the teachings of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention exploits the dynamics of harmonic oscillators for tissue characterization. For the purpose of providing a complete and self contained description of the invention, an introductory explanation of the principles of harmonic oscillators precedes the detailed description of the invention in context of the drawings describing its preferred embodiments.

Many systems in nature which vibrate or oscillate may be approximated by a well known physical model, called harmonic oscillator. A simple harmonic oscillator is a physical system in which a generalized coordinate representing the system is proportional to its second derivative, where the constant of proportionality is negative. The generalized coordinate of the system may be realized as, for example, a displacement, an angle, an electric charge or any other degree-of-freedom in the system.

Mathematically, a harmonic oscillator is represented by one or more differential equations, called the equations of motion. The number of equations of motion depends on the number of generalized coordinates, and the solutions of these equations describe the functional dependence of the generalized coordinates on time. The solution of a simple harmonic oscillator is a periodic function characterized by a frequency, called the natural frequency. The natural frequency depends on the parameters of the system, which parameters are referred to as the constant of inertia (or the inertia) and the elastic constant (or the elasticity).

The most illustrative example of a harmonic oscillator is a mass connected to some elastic object of negligible mass (e.g., a spring) that is fixed at the other end and constrained so that it can only move in one dimension. In this example, the generalized coordinate may be the position of the mass, the constant of inertia is the mass and the elastic constant is the spring constant measured in units of force per mass unit. Another typical example of harmonic oscillator is an electric circuit which comprises a capacitor and an inductor. In this example the generalized coordinate is the electric charge on the capacitor, the constant of inertia is the inductance and the elastic constant is related to the capacitance. Other examples include a pendulum on a long wire, a molecule, motion of a charged particle within a quadratic potential (e.g., inside a spherically symmetrical charged distribution), acoustic waves and the like. Irrespective of the physical realization of the system, all the mathematical solutions of harmonic oscillations are equivalent.

For each harmonic oscillator, there is one particular point in which, under certain initial conditions, no oscillations occur. This point is referred to as the point of equilibrium. For example, for a mass connected to a horizontal spring, the point of equilibrium is the point where the spring is loose. When the system is displaced from its equilibrium position, the elasticity provides a restoring force which is directed to the equilibrium position, and the inertia property causes the system to overshoot equilibrium. A continued interplay between the elastic and inertia properties of the system results in an oscillatory motion. In a simple harmonic oscillator, the motion is characterized by a natural frequency which is related to the elastic constant and constant of inertia.

In reality, dissipative forces prevent from the simple harmonic oscillator from its perpetual motion, and, unless other driving forces exist, the oscillations of the system decrease with time. Such a physical model is called a damped harmonic oscillator, and the decreasing rate of the oscillations depends on other parameters of the system. In terms of energy, a damped harmonic oscillator releases energy to the environment, typically by heating the medium in which the system oscillates. Once all the energy of the system is released, the system is loose again. An approximation to the dissipative force resulting from friction between the harmonic oscillator and the medium in which it oscillate, is a force which is proportional to the velocity of the system where the constant of proportionality, called the damping factor, is negative. In this case, the amplitude of the oscillations decreases exponentially with time.

A time-dependent external force, acting on a damped harmonic oscillator, may compensate the energy lose of the system so that the system continues to oscillate while still subjected to the dissipative force. This case is referred to as a damped and forced harmonic oscillator, or damped and driven harmonic oscillator. When the dissipative force is proportional to the velocity of the system and the driving force oscillates in a sinusoidal manner, the equation of motion of the system has an analytic solution consisting of two parts: a transient part and a steady-state part. The transient part is characterized by an amplitude which depends on the initial conditions of the system and corresponds to a damped harmonic oscillator, i.e., decreases exponentially with time. The steady-state part is characterized by a constant amplitude that depends on the driving force, but does not depend on the initial conditions of the system. The amplitude of the steady-state part depends on the relation of the frequency of the driving force to the natural frequency of the system and on the damping factor.

A particular case in which the driving frequency equals the natural frequency is called a resonance. The maximal value of the steady-state amplitude occurs at a driving frequency smaller than the resonance frequency (for constant driving force amplitude). As the damping factor decreases, the maximal amplitude frequency tends to the resonance frequency value, and the amplitude increases as the reciprocal of the damping factor. A frequency response curve is a graph representing the steady-state amplitude as a function of the driving frequency. Typically, the response curve has a sharp peak near the resonance frequency. Hence, by scanning the driving frequency of a damped and forced harmonic oscillator, one can locate the resonance frequency of the system and gain information on the system in general and on its parameters in particular.

Based on Hooke's law, all materials in nature, including tissues, have some elastic properties and in certain deformation regions may be viewed as harmonic oscillators.

While conceiving the present invention it was hypothesized that the mechanical properties of tissues may be measured by studying the response curve of the tissues-of-interest. It has been further hypothesized that based on the mechanical properties, the nature of a tissues-of-interest may be characterized.

Thus, according to one aspect of the present invention there is provided a system for characterizing a tissue present in a predetermined location of a body of a subject, generally referred to herein as system 10. The tissue may be any tissue which can be characterized according to its mechanical properties, e.g., a tumor (malignant or benign), a blood vessel (e.g., stenotic tissue, wall tissue, plaque), a bone, a pathological tissue or any other a part of an organ (either internal or external). System 10 can be used to characterize a tissue in a location which has already been determined by another medical procedure, e.g., an ultrasonic imaging procedure, MRI and the like and provides another dimension to diagnostic procedures.

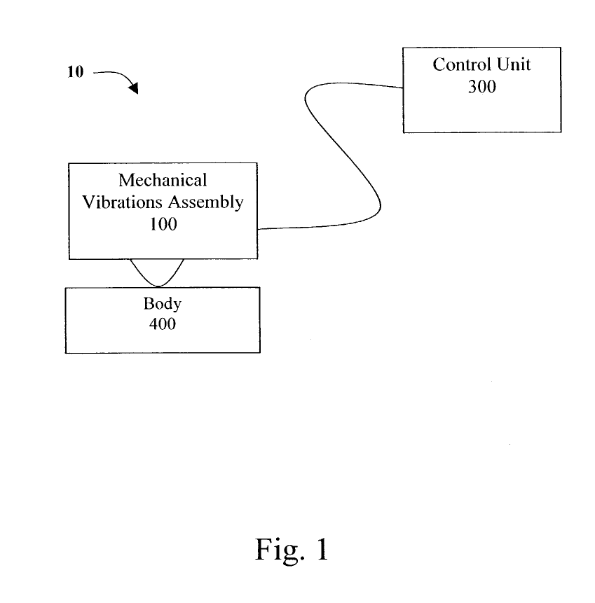

Referring now to the drawings, FIG. 1 illustrates system 10 which comprises a mechanical vibrations generating assembly 100, and a control unit 300. In use, mechanical vibrations generating assembly 100 generates mechanical vibrations at a position adjacent to the predetermined location of body 400. Hence, assembly 100 serves for supplying the oscillating driving force to the system, as explained hereinabove.

According to a preferred embodiment of the present invention control unit 300 serves two purposes: (i) scanning the driving frequency of the mechanical vibrations generated by assembly 100; and (ii) measuring a frequency response spectrum from the predetermined location. Thus, control unit 300 communicates with assembly 100 in a manner that signals from control unit 300 are converted into the vibrations of assembly 100, and signals from assembly 100 are converted into readable data by control unit 300. The frequency range in which system 10 operates is preferably 10-10000 Hz, more preferably 15-5000 Hz, still preferably 20-5000 Hz most preferably 20-2500 Hz.

According to a preferred embodiment of the present invention the mechanical vibrations are applied onto the skin, thereby provide mechanical excitations of the skin near the predetermined location which is to be characterized. As is further detailed hereinafter, the data, collected by control unit 300, which reflect excitation of body 400 at the external point of contact is sensitive to the mechanical properties of the tissue deep inside the body. In other words, it will be demonstrated that mechanical properties of internal tissues are characterized by external measurements.

According to this embodiment of the invention, system 10 is non invasive. Nevertheless, the scope of the present invention is not limited to non invasive systems and, as further detailed hereinbelow, it will be appreciated that systems operable similar to system 10, yet can be adapted for use in minimally invasive (e.g., catheter based) and more invasive procedures (e.g., during invasive operation) are also within the scope of the present invention.

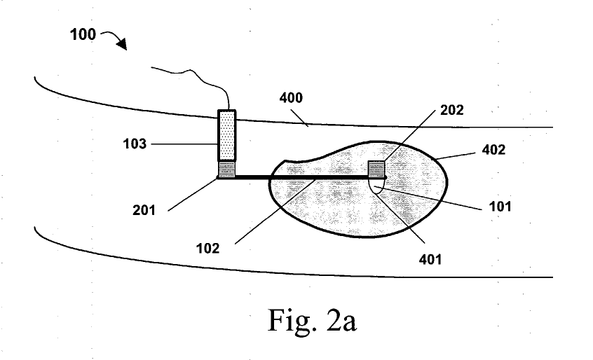

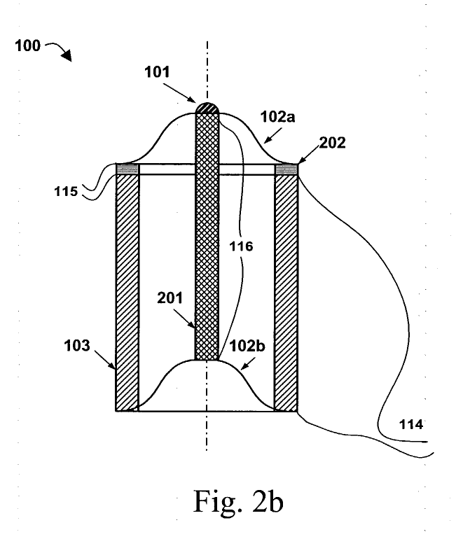

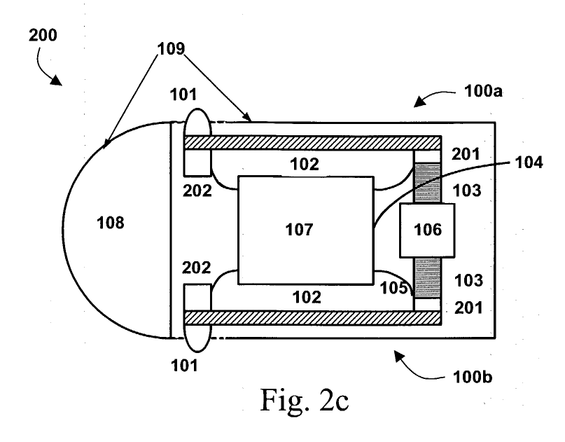

Reference is now made to FIG. 2a, which illustrates a typical configuration of assembly 100 operating on a body 400.

According to a preferred embodiment of the present invention, assembly 100 includes a Mechanical Linkage Device (MLD) 102, which serves for transferring mechanical vibrations to body 400.

MLD 102 is in contact with body 400 (for example, at position 401 shown in FIG. 2a), preferably through a contact-tip 101. According to a preferred embodiment of the present invention, in addition to the application of the driving force, MLD 102 may also be used to measure the displacement (e.g., of position 401), with minimal distortions. Being an object which dynamically interacts with body 400, MLD 102 substantially improves the capability of system 10 to distinguish between different biological materials inside the body.

MLD 102 may be, for example, an elastic rod, a leaf spring, a system of springs and masses or any other device which is capable of applying the driving force to body 400. According to a preferred embodiment of the present invention MLD 102 is made of a soft and light material so as to allow MLD 102 to exert a substantially constant force amplitude, e.g., at position 401. In addition, MLD 102 is characterized by a natural frequency which is preferably higher than the frequency of the driving force, so as to minimize dynamical distortion. A judicious selection of the size and the natural frequency of MLD 102 increases the dynamical interaction between the body and the MLD, thus allows for the distinction between different biological materials.

As stated, contact-tip 101 provides for the physical contact between system 10 and the body. Contact-tip 101 may be of any shape suitable to convey the vibrations generated by assembly 100 into the body. Preferably, contact-tip 101 is sterile. Sterilization can be achieved, for example by providing a sterilizable cover onto contact-tip 101, or by manufacturing it from disposable (sterilizable) material, so that it can be replaced between successive operations of system 10.

Several contact-tips, positioned in more than one position adjacent to the predetermined location, may also be used. Contact-tip 101 may be in position 401 adjacent to the tissue which was detected using a previous medical imaging procedure (e.g., ultrasonic, magnetic resonance or x-ray imaging). However, in some cases, an exact location is not known since the medical imaging apparatus only provides a suspected area 402. In this case, contact-tip 101 may be moved or scanned to other positions 402, so as to optimize the measurement.

The orientation of contact-tip 101 with respect to body 400 is determined by the user in accordance with the desired direction of the applied mechanical vibrations. For example, in one embodiment, the vibrations are perpendicular to the plane of body 400, constraining mechanical excitations of the molecules normal to the skin. In another embodiment, the vibrations are inclined to body 400 by a predetermined inclination angle (e.g., 10-80 degrees), allowing for mechanical excitation vectors being both normal and parallel to body 400.

The procedure may also be repeated a plurality of times, where in each time contact-tip 101 engages a different position and/or inclined by a different inclination angle, and the resulting measurements may be analyzed simultaneously and/or independently.

To facilitate multiple measurements, and according to another embodiment of the present invention, a plurality of contact-tips 101 arranged in a matrix-like arrangement are used for simultaneous detection from a plurality of positions and/or a plurality of inclination angles, obviating or reducing the need for scanning the positions/angles for optimum. An aspect ratio of the matrix is preferably selected so as to allow a substantial efficient scanless measurement of body 400.

According to a preferred embodiment of the present invention, the positioning and/or orienting of contact-tip 101 is carried out either manually or automatically, hence, assembly 100 may be manufactured sufficiently compact to facilitate mobility of system 10, or it may include by a suitable machinery for moving contact-tip 101 from one location to another and/or for varying its inclination angle.

According to another embodiment of the present invention, assembly 100 may also include a mechanism for isolating assembly 100 from environmental vibrations.

This may be for example a stand or any other apparatus having static parts attached to a fixed point (e.g., floor, ceiling or wall) and non static parts which can move freely and independently from the static parts. Preferably, the motion of the non static parts is both translational motion and rotation motion. More preferably, the translational motion is governed by three degrees-of-freedom. Still preferably, rotation motion is governed by at least two rotational degrees-of-freedom.

Referring again to FIG. 2a, in a preferred embodiment of the invention, assembly 100 further comprises a mechanical vibrations generating transducer assembly 103 operable to convert electrical signals from control unit 300 into mechanical motions, e.g., vibratory motions. Transducer 103 may operate using any principles known in the art, such as, but not limited to, piezoelectric, electric, electrostrictive, magnetic, magnetostrictive, electromagnetic, micro electro mechanical system (MEMS), or electrostatic principles.

Preferably, assembly 100 further comprises at least one mechanical sensor. A mechanical sensor is a device for converting mechanical signals (acceleration, force, pressure, displacement, etc.) into electric signals. Two mechanical sensors are shown in FIG. 2a, a first sensor 201 coupled to transducer assembly 103, and a second sensor 202, coupled to contact-tip 101. It is to be understood, however, that more sensors may be included in assembly 100, to better facilitate data acquisition. The sensors may be either contact sensors or remote sensors. In the example given, first sensor 201 serves for sensing the vibrations as transmitted from transducer assembly 103. Preferably, sensor 201 is a force sensor that is used to control the transducer assembly 103 via control 300 to emit constant force versus frequency. Second sensor 202 serves for sensing the mechanical response from the body, as manifested by the motion of contact-tip 101. Both first 201 and second 202 sensors communicate with control unit 300, as further detailed hereinunder.

A particular feature of a preferred embodiment of the present invention is that second sensor 202 is coupled to contact tip 101. This feature has the advantage that the number of contact points between system 10 and the subject is minimized (e.g., one contact point). However, it is intended not to limit the scope of the present invention for use of any specific configuration of sensors hence other alternatives may be used. For example, sensor 202 may be attached to the body of the subject substantially near position 401, or, a plurality of sensors 202 may be attached to the body at different positions within area 402. In any case, sensor(s) 202 electrically communicates ( e.g., by an appropriate wiring setup), with control unit 300.

According to a preferred embodiment of the present invention assembly 100 may comprise more than one MLD, so as to improve the operation of system 10.

Reference is made to FIG. 2b, which is a cross sectional view of assembly 100, in the embodiment in which more than one MLD is used. Two MLDs are shown in FIG. 2b, a first MLD, designated 102a and a second MLD designated 102b.

In this embodiment, transducer 103 has a tubular shape, where first MLD 102a is positioned on one end of transducer 103 and second MLD 102b is positioned on another end of transducer 103. according to a preferred embodiment of the present invention transducer 103 may be any transducer of tubular shape which is capable of transforming electrical signal into a mechanical signal and may operate according to any known principle as further detailed hereinabove, for example, a tubular electromagnetic coil, a toroidal electromagnetic coil, a piezoelectric tube, a piezoelectric annulus, a piezomagnetic tube and the like.

First sensor 201 is preferably elongated (e.g., shaped as rod), and positioned so as to connect first MLD 102a and second MLD 102b. First 102a and second 102b MLDs are preferably identical thin membranes (e.g., from thin plastic or thin metal, provided that transducer 103 and first sensor 201 are electrically insulated from each other). Sensor 201 serves for receiving mechanical input from tip 101 which, in operational mode of assembly 100, is continuously in contact with body 400. Sensor 201 may be any sensor capable of transforming an axial mechanical signal into an electrical signal, such as, but not limited to, a piezoelectric rod, a tubular electromagnetic coil, a piezomagnetic.

In another preferred embodiment, MLDs 102a and 102b may be made of piezoelectric polymeric membranes, so as to serve also as sensors. The advantage of such configuration is that the sensing functionality is intrinsic to MLDs 102a and 102b, so that the part, designated in FIG. 2b by numeral 201, may be a connection rod rather then a sensor. As such, the connection rod (201) may be made of any hard material, e.g., metal or plastic. In this embodiment, the output of MLDs 102a and 102b to control unit 300 is by leads 116 and 114.

Second sensor 202 serves as a monitor of transducer 103. According to a preferred embodiment of the present invention, the shape of second sensor 202 matches the shape of transducer 103 so as to allow sensor 202 to measure the vibrations of transducer 103. For example, for a cylindrical shape of transducer 103 sensor 202 may an annulus. Sensor 202 may be for example, a force sensor, an accelerometer, a displacement sensor and the like.

In operational mode, control unit 300 sends input signals to transducer 103 (e.g., via a lead 114 connected thereto), and monitors transducer 103 output using second sensor 202 that is connected to the control unit 300 by cables 115. Transducer 103 transfers the electrical input signals into mechanical input signals which are transferred from transducer 103 to contact tip 101 via MLD 102b, sensor 201 and MLD 102a. Contact tip 101 vibrates in response to the mechanical input signals and sensor 201 measures these mechanical response vibrations, transforms these vibrations into electrical signals and transmits these signals back to control unit 300 (e.g., via a lead 116 connecting sensor 201 and control unit 300). According to a preferred embodiment of the present invention, first MLD 102a and second MLD 102b substantially prevent first sensor 201 from any motion mode other than axial mechanical vibrations as picked up by tip 101. Undesired motion modes of first sensor 201, which may be prevented by MLDs 102a and 102b include, but are limited to, bending, buckling, twisting and the like.

As stated, system 10 may also be adapted for use in minimally invasive and more invasive procedures. According to a preferred embodiment of the present invention assembly 100 may be designed and constructed so as to operate inside a tube where tip(s) 101 touches the inner surface of the tube at one or more points. With such design, assembly 100 may be, or may be mounted on an endoscopic probe to be inserted into the vascular, cardio-vascular or urinary system of a mammal.

According to a preferred embodiment of the present invention one or more assemblies may be combined with additional imaging devices to form an endoscopic device 200, which is schematically illustrated in FIG. 2c.

Referring to FIG. 2c, device 200 may comprise several mechanical vibrations generating assemblies (such as assembly 100), arranged in an encapsulation 109 having a sufficiently small diameter so as to allow motion of device 200 in the mammalian vascular, cardio-vascular or urinary system. To simplify the following description, two assemblies are shown in FIG. 2c, designated 100a and 100b. It is to be understood, however, that this should not be considered as limiting and any number of assemblies may be used. Additionally, as described herein, device 200 operates as a part of system 10, and, as such, being in communication with control unit 300, via lead 104. It is to be understood that device 200 may also be used with other systems provided these system can communicate therewith. For example, device 200 may be combined with an endoscopic system being used for the various minimal invasive treating procedures of the vascular, cardio-vascular or urinary system.

Assemblies 100a and 100b may be configured in more than one way, provided that mechanical vibrations are transmitted thereby to the respective position of body 400. More specifically, each of assemblies 100a and 100b may independently be manufactured as described hereinabove with reference to FIGS. 2a and 2b. Without limiting the scope of the present invention, and for illustrative purposes only, assemblies 100a and 100b which are shown in FIG. 2c are similar to assembly 100 shown in FIG. 2a.

Device 200 comprises at least one imaging device 108, such as, but not limited to, an Intra Vascular Ultra Sound (IVUS) device, Intra Vascular Magnetic Resonance (IVMR) device, a camera or any other imaging device suitable for being integrated into an endoscopic probe. Alternatively, imaging device 108 may be located outside device 200 in a manner that allows imaging device 108 to communicate with device 200, for example, via optical (e.g., infrared, visible, ultraviolet), electrical, or acoustical communication channel. In this embodiment, imaging device 108 may also be a noninvasive imaging device, such as, but not limited to, a computer tomography device or a magnetic resonance device.

Imaging device 108 serves for initial detection of the region to be analyzed by assemblies 100a and 100b (and additional assemblies which, as stated, may be present in device 200).

In operational mode device 200 moves, e.g., within a blood vessel in a manner that tips 101 and MLDs 102 of assemblies 100a and 100b are contracted towards the inner part of device 200. When imaging device 108 detects a region-of-interest (e.g., a region having a suspected plaque or other vascular sediments), device 200 stops as to juxtapose at least one of tips 101 opposite to the region-of-interest. Alternatively, if the region-of-interest is farer from device 200 assembly, a posing mechanism 106 bulges tip(s) 101 (and, if necessary also MLD(s) 102) out of encapsulation 109 so as to touch the tissue of the region-of-interest.

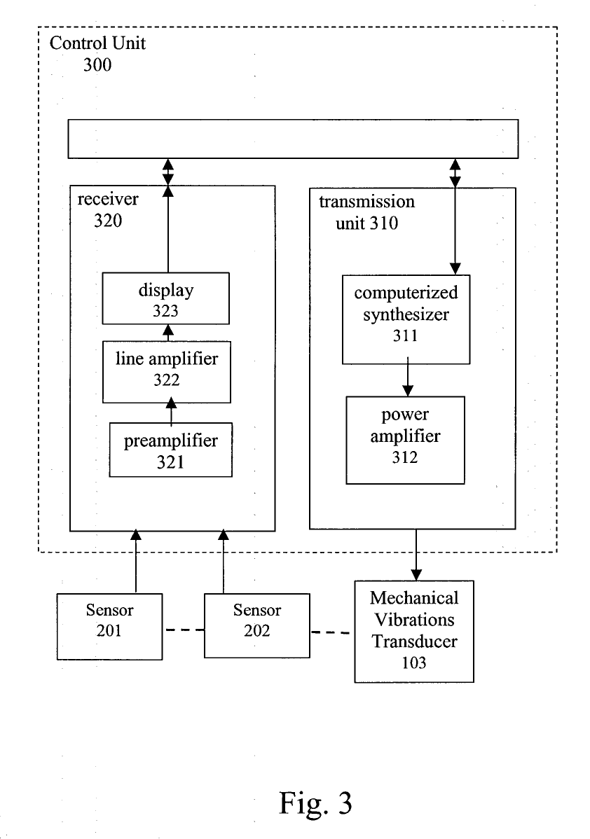

Once a contact has been established between tip 101 and the suspected tissue, transducer 103 sends mechanical signals to, and receives responses of tip 101, via MLD 102. If more than one tip touches the suspected tissue, the mechanical signals are preferably transmitted to each of the operative tips, as further detailed hereinabove. The mechanical responses are then used for the analysis of the suspected tissue (e.g., by control unit 300 as further detailed hereinunder, with reference to FIG. 3).

Once a certain region-of-interest is analyzed, mechanism 106 withdraws tip 101 and MLD 102 back into encapsulation 109 so as to facilitate a substantially free motion of device 200 to the next region-of interest.

According to a preferred embodiment of the present invention device 200 further comprises a preamplifier 107 electrically communicating with sensors 201 and 202, (e.g., via leads 105) for partial amplifying of the electrical signals received from sensors 201 and 202. The partial amplification of the electrical signals is particularly useful for improving the efficiency of data analysis. Specifically, as device 200 is essentially far from control unit 300, a partial amplification, prior to the transmission of the signals to control unit 300 increases the signal-to-noise ratio thereby improves the accuracy of the measurement.

Reference is now made to FIG. 3 which illustrates control unit 300, according to a preferred embodiment of the present invention. Control unit 300 comprises a transmission unit 310 a receiver 320 and a processor 330. In FIG. 3, electrical communication channels are shown as solid arrows, where the directions of the arrows indicate information flow, and mechanical linkages are shown as dashed lines. Transmission unit 310 serves for transmitting an electrical signal to assembly 100, receiver 320 serves for receiving an electrical signal from assembly 100 and processor 330 serves for controlling the electrical signals to be transmitted from transmission unit 310, and for analyzing the electrical signals as collected by receiver 320. Specifically, processor 330 serves for sampling control, data acquisition, data recording, data analysis and for displaying the results of the measurements.

According to a preferred embodiment of the present invention transmission unit 310 comprises a computerized synthesizer 311 for generating a synthesized electrical pulse, synthesizer 311 communicates with processor 330. Transmission unit 310 further comprises a power amplifier 312 for amplifying the electrical pulses, prior to the transmission of the pulses to transducer assembly 103. Transmission unit 310 communicates with transducer assembly 103.

According to a preferred embodiment of the present invention receiver 320 comprises a preamplifier 321 and a line amplifier 322 which are configured and designed to amplify the electrical pulses received from assembly 100. In addition, receiver 320 comprises a display 323 for displaying the electrical pulses. Display 323 may be an oscilloscope, a spectrum analyzer, a computer display, a printer or any other known suitable device. First sensor 201 and second sensor 202 are operable to send electrical signals to receiver 320 so as to allow measurement of the relation between the amplitude of the driving force and the response amplitude.

The electrical pulses from transmission unit 310 which are controlled by processor 330 determine the frequency of the mechanical vibrations applied to the body by MLD 102.

According to a preferred embodiment of the present invention the electrical pulses are selected so as to enhance the mechanical excitations of the tissue and thereby the quality of the measurement. Hence, the mechanical vibration frequency may be, for example, a single frequency, a superposition of a plurality of frequencies, a continuous frequency scan (chirp) or a band-limited white noise frequency, depending on the examined tissue and/or the sensitivity of the equipment which is used in the various embodiments of the invention as is further detailed hereinabove.

According to another aspect of the present invention there is provided a method of characterizing a tissue present in a body of a subject. The tissue undergoing analysis using the method of the present invention can be any of the tissues, either normal or pathological as is further detailed hereinabove. Prior to the characterization of the tissue, the location of the tissue may be determined by another diagnostic, e.g., imaging device, e.g., an ultrasonic imaging device.

The method of this aspect of the present invention comprises the following method steps, in which in a first step mechanical vibrations adjacent to the predetermined location of the tissue are generated. Preferably, the first step is executed so as to optimize the measurement (i) by minimizing effects of environmental noise occurring while the mechanical vibrations are applied, and (ii) by selecting an appropriate position and/or direction of the mechanical vibrations, as further described hereinabove. In a second step, a frequency of the mechanical vibrations is scanned, and in a third step a frequency response spectrum is measured, so as to obtain at least one mechanical property of the tissue.

Each of the above method steps can be carried out using an appropriate equipment or machinery. For example, the first step may be executed using a vibrator, the second step may be executed by varying the power supply of the vibrator and the third step may be executed by a system of sensors which are controlled by a central data processor. Alternatively, one or more of the above method steps may be executed by system 10, as described above.

The present invention provides a method and a system which successfully characterize a large variety of tissues, present in a predetermined location in the body. The position onto which the vibrations are applied (e.g., the position of contact-tip 101) is determined by the type and location of the tissue-of-interest, as further detailed herein. Thus, in cases where the tissue forms a part of, or is associated with, a blood vessel tissue, e.g., forms a plaque inside a blood vessel, the preferred position of contact-tip 101 is onto the skin which is closest to the blood vessel-of-interest, e.g., closest to the carotid, one of the femoral vessels or the abdominal aorta, and the like. In cases where the tissue is a lesion (either a dermal lesion, a sub-dermal lesion or an internal lesion), the preferred position of contact-tip 101 is onto the skin which is closest to the lesion. Lesions include, for example, melanoma, breast cancer, cancer of the prostate and the like.

It will be recognized that, in order to allow for efficient therapeutic procedures to be practiced, melanoma, for example, must be positively diagnosed malignant in phase I (skin surface) or II (up to 3-4 mm deep), both of which are within the scope of the present invention.

In cases of breast or prostate cancer the lesion is located at a small depth (several centimeters) below the outer surface of the skin. Therefore the preferred position of contact-tip 101 is onto the breast or lower abdomen.

In cases where the tissue is a bone (such as, but not limited to, a tibia or fibula), the preferred position of contact-tip 101 is onto the skin which is closest to the bone (e.g., on the leg of the subject).

In other cases the tissues-of-interest is in the lungs (for example, when the lungs are inflamed, suffer an edema or any other fluid fill or are suspected of lung malignancy) the preferred position for contact-tip 101 is onto the thorax.

As stated, the information gained from the mechanical property of the tissue is sufficient for characterizing and identifying the tissue-of-interest. Nevertheless, tissue characterization, according to the present invention, can be done in more than one way.

In one embodiment, the frequency response spectrum is used for calculating at least one mechanical property of the tissue.

Preferably, the calculated mechanical properties are elastic constants, e.g., an elastic modulus, a Poisson's ratio, a shear modulus, a bulk modulus or a first Lamé coefficient. One ordinarily skilled in the art would appreciate that for isotropic materials, it is sufficient to measure two of the above elastic constants and then to calculate the other using theoretical formulae. Such formulae are available for example in a text book by Timoshenko & Young, entitled “Theory of elasticity”, which is incorporated by reference as if fully set forth herein.

In another embodiment of the invention, the frequency response spectrum is compared to an existing database (e.g., a library having a plurality of resonance spectra for different types of tissues). Such a comparison can be executed on, for example, normalized spectra using, for example, a simple square minimal error (SME) mathematical procedure. Other procedures and manipulations of the data, such as, but not limited to, correlation, transfer functions, coherence and cepstrum are not excluded.

To this end, according to yet another aspect of the present invention there is provided a method of constructing a resonance spectra library, the resonance spectra characterizing a plurality of tissues of a plurality of subjects. The method comprising the following method steps, in which, in a first step a tissue of a subject is selected and mechanical vibrations are generated at a position adjacent to the tissue. As will be explained below, the selected tissue is to be associated with the frequency response spectrum. In a second step of the method, a frequency of the mechanical vibrations is scanned, in a third a frequency response spectrum from of the tissue is measured, and a forth step comprises recording the frequency response spectrum, thereby providing a frequency response spectrum entry of the library, which entry characterizes the selected tissue.

Hence, for each tissue one or more entries are recorded, thereby a resonance spectra library is constructed. Entries (e.g., normalized spectra) from similar tissues can be averaged. According to a preferred embodiment of the present invention each of the steps of this aspect of the invention may be executed by any known equipment or machinery, for example, by system 10. It is to be understood that the steps of this method may be repeated a plurality of times, each time for different tissue of the same subject and/or for different subject, so as to increase the size, representability and/or accuracy of the resonance spectra library.

Once constructed, the resonance spectra library can be stored in an appropriate memory media for future use, e.g., by system 10 or by other aspects of the present invention as describe above.

Hence, according to yet an additional aspect of the present invention there is provided a resonance spectra library produced, as detailed hereinabove, by the method. The resonance spectra of the library are preferably stored, in a retrievable and/or displayable format, on a memory media.

According to still an additional aspect of the present invention there is provided a memory media, storing in a retrievable and/or displayable format the resonance spectra of the resonance spectra library.

According to a preferred embodiment of the present invention the memory media can be any memory media known to those skilled in the art, which is capable of storing the resonance spectra library either in a digital form or in an analog form. Preferably, but not exclusively, the memory media is removable so as to allow plugging the memory media into a host (e.g., a processing system), thereby allowing the host to store the resonance spectra library in it or to retrieve the resonance spectra library from it.

Examples for memory media which may be used include, but are not limited to, disk drives (e.g., magnetic, optical or semiconductor), CD-ROMs, floppy disks, flash cards, compact flash cards, miniature cards, solid state floppy disk cards, battery-backed SRAM cards and the like.

According to a preferred embodiment of the present invention, the resonance spectra library is stored in the memory media in a retrievable format so as to provide accessibility to the stored data. Preferably information is retrieved from the resonance spectra library either automatically or manually. That is to say that the resonance spectra library may be searched by an appropriate set of search codes, or alternatively, a user may scan the entire library or a portion of it, so as to find a match for the measured frequency response spectrum. According to a preferred embodiment of the present invention the resonance spectra library is stored in the memory media in more than one form.

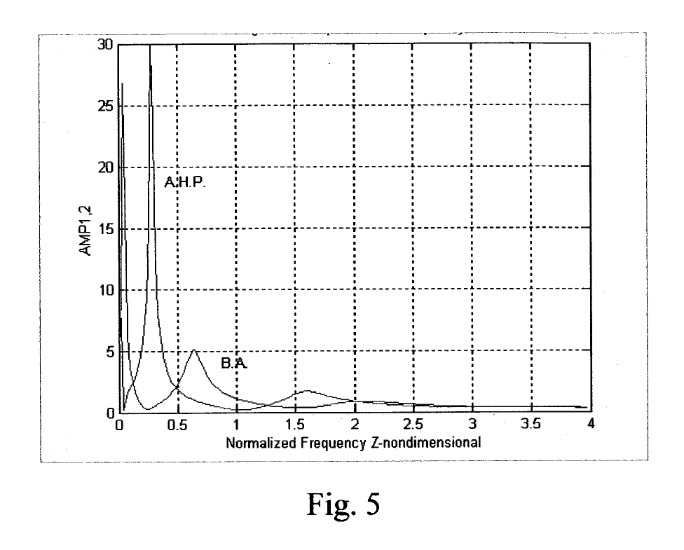

Hence, in one embodiment the library includes a plurality of images which may be compared the measured resonance curve. Examples for images which may be stored in the library are given in FIGS. 5-12, 15-22, 24 and 25 which are further discussed in the Examples section below.

In another embodiment, the resonance spectra of the library are stored in a textual format which facilitates searching the library using search codes. For example the library may contain elastic moduli of several tissues or the library may contain normalized amplitudes an&or normalized phase angles as a function of normalized frequencies, as further detailed in the Examples section hereinunder.

It is appreciated that in all the above embodiments, library data is stored in the memory media in an appropriate displayable format, either graphically or textually. Many displayable formats are presently known, for example, TEXT, BITMAP™, DIF™, TIFF™, DIB™, PALETTE™, RIFF™, PDF™, DVI™ and the like. However it is to be understood that any other format that is presently known or will be developed during the life time of this patent, is within the scope of the present invention.

In addition to the resonance spectra library, to which each measured spectrum can be compared, so as to identify the type of tissue, the characterization of the tissue, or any other structural material, may be done also by simulating one or more harmonic oscillators.

Thus, according to still another aspect of the present invention there is provided a method of classifying a frequency response spectrum of a structural material. The method is executable by a data processor and comprising the following method steps, in which, in a first step, a physical model of a plurality of harmonic oscillators is constructed. The physical model may be of any number of dimensions and independently of any number degrees-of-freedom, it comprises a set of parameters and it is characterized by a plurality of equations of motion. The set of parameters may be, for example, one or more constants of inertia (e.g., mass or inductance) and one or more elastic constants (e.g., spring constant or reciprocal of capacitance). A skilled artisan would appreciate that the set of parameters described herein represent dynamic stiffness and density of the structural material which is to be classified.

According to a preferred embodiment of the present invention at least one of the harmonic oscillators is a damped harmonic oscillator and at least one of the harmonic oscillators is a forced harmonic oscillator; hence, the physical model is characterized by at least one driving frequency.

A second step of this method of the present invention is to simultaneously solve the plurality of equations of motion, so as to provide at least one frequency response, which may be, for example, a frequency dependent amplitude or a frequency dependent phase. Examples of physical models and solutions are given in the Examples section below.

In a third step of the method, a frequency response is compared with the frequency response spectrum of the structural material. The comparison may be done by checking overlaps between curves or by numerical comparison. The first two steps of this aspect are preferably repeated, each time with a different set of parameters, while each time the frequency response is compared with the frequency response spectrum of the structural material. Once an appropriate set of parameters that matches the frequency response spectrum is found, the frequency response spectrum of the structural material is classified based on the particular set of parameters.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples which, together with the above descriptions, illustrate the invention in a non limiting fashion.

### Example 1

### One Dimensional Model

The body is a continuous mass system with viscoelastic properties. The present example is a one dimensional model of a certain region of the body. The model comprises a system of a plurality of degrees-of-freedom each degree-of-freedom is constrained to a one dimensional motion.

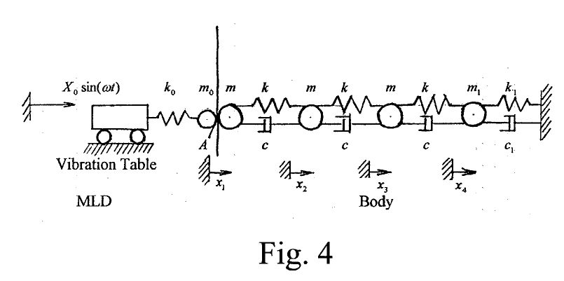

FIG. 4 illustrates the system where each degree-of-freedom is represented by a displacement, x, mass, m, connected to a spring having a spring constant, k, and is subjected to a dissipative force having a damping factor, c. The leftmost mass of the system is connected to a Mechanical Linkage Device (MLD), consisting of a soft spring, k₀, a small mass, m₀, and a table which vibrates harmonically with frequency ω. Hence, the model is a one dimensional many degree of freedom, damped and forced harmonic oscillator.

The degrees-of-freedom of the system represent the mass lumped parameters of the body, where the rightmost mass represents an arterial tissue which is to be characterized. As the model is directed for simulating a non invasive procedure, the observable is the particle which is close to the surface of the body, i.e., the mass which is in contact with the MLD. The contact point is designated A in FIG. 4. The displacements of the masses are denoted by xᵢ, i=1, . . . , 4, and each time derivative is denoted by a dot above the corresponding displacement (e.g., [symbol] dx/dt and [symbol] d²x/dt²).

The equations of motion of the system are:

$$\begin{matrix}( {m}_{0} + {m}_{1} ) {x}_{1} = - k ( {x}_{1} - {x}_{2} ) + {k}_{0} ( {X}_{0} sin ( ω t ) - {x}_{1} ) - c ( {x}_{1} - {x}_{2} ) \\ {mx}_{2} = - k ( {x}_{2} - {x}_{1} ) - k ( {x}_{2} - {x}_{3} ) - c ( {x}_{2} - {x}_{1} ) - c ( {x}_{2} - {x}_{3} ) \\ {mx}_{3} = - k ( {x}_{3} - {x}_{2} ) - k ( {x}_{3} - {x}_{4} ) - c ( {x}_{3} - {x}_{2} ) - c ( {x}_{3} - {x}_{4} ) \\ {m}_{1} {x}_{4} = - {k}_{1} {x}_{4} - k ( {x}_{4} - {x}_{3} ) - {c}_{1} {x}_{4} - c ( {x}_{4} - {x}_{3} )\end{matrix} \qquad ( EQ . 1 )$$

The natural vibration of the system decays due to the dissipative forces, and the steady-state solution to Equation 1 is obtained by the following substitution:

xᵢ=Aᵢ sin(ωt)+Bᵢ cos(ωt),i=1,2,3,4 (EQ. 2)

The result is a set of 8 linear equations, the solution of which yields the 8 constants Aᵢ, Bᵢ.

For the point of contact (representing response of a particle on the surface of the body), the vibration amplitude is \sqrt{A₁²+B₁²} and the phase angle is φ=tan⁻¹(B₁/A₁).

The set of parameters of the model are the masses and the spring constants. For normal arterial tissue, m₁=m and k₁=0.1k, where small spring constant corresponds to a soft arterial tissue compared to a tissue adjacent to the artery. On the other hand for a malignant tissue such as a hard plaque which is added onto the artery, the mass is large (m₁=10m). For stiffened artery the spring constant is larger than the spring constant of a normal artery (k₁=k).

Reference is now made to FIGS. 5-8 showing a comparison between a benign arterial tissue which has been calculated using the relations: m₁=m and k₁=0.1k, and a hard plaque tissue, which has been calculated using the relations m₁=10m and k₁=0.1k.

Curves on FIGS. 5-8 which are designated by the letters AHP correspond to calculations for added hard plaque, and curves which are designated by the letters BA correspond to calculations for benign artery. FIG. 5 shows a normalized amplitude, AMP, as a function of a normalized frequency, Z. Both quantities are non-dimensional and defined as:

$$\begin{matrix}AMP = \sqrt{{A}_{1}^{2} + {B}_{1}^{2}} \times \frac{k}{{k}_{0} {x}_{0}} & ( EQ . 3 ) \\ Z = \frac{{ω}^{2}}{k m} . & ( EQ . 4 )\end{matrix}$$

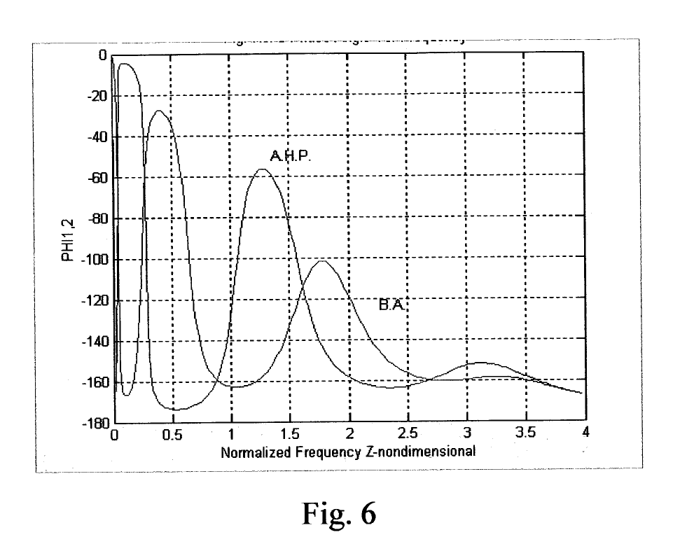

FIG. 6 shows the phase angle, φ, which is designated on the plot as PHI, as a function of the normalized frequency, Z.

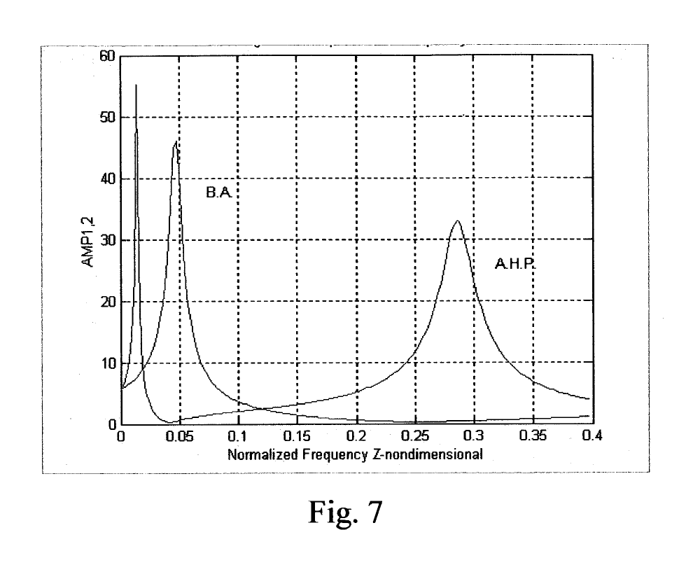

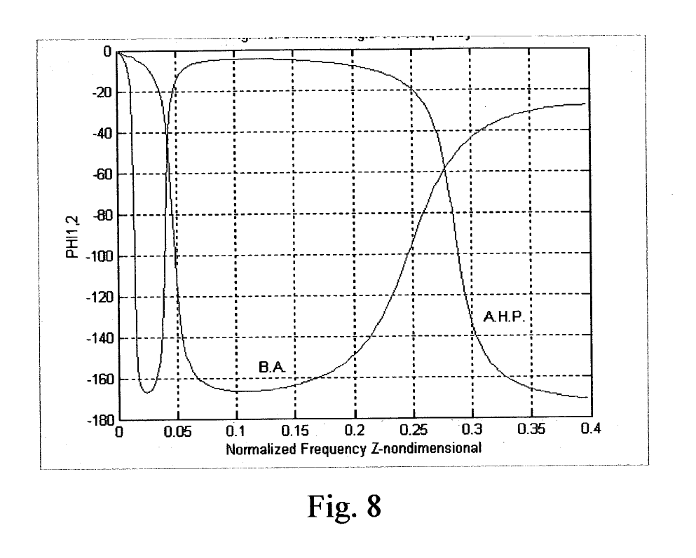

FIGS. 7-8 show, respectively, the normalized amplitude and the phase angle, as a function of the normalized frequency, for low normalized frequencies. The resonance frequencies for the benign artery were observed at: Z=0.04, 0.64 and 2.08. The forth resonant was attenuated completely by the friction

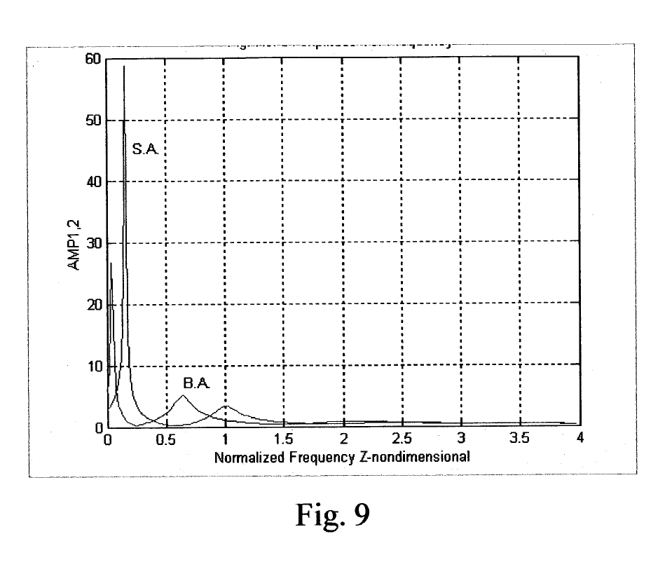

Reference is now made to FIGS. 9-12 showing a comparison between the benign arterial tissue, and stiffened arterial tissue, which has been calculated using the relations m₁=m and k₁=k. Curves on FIGS. 9-12 which designated by the letters SA correspond to calculations for stiffened artery. Benign artery curves are still designated by the letters BA.

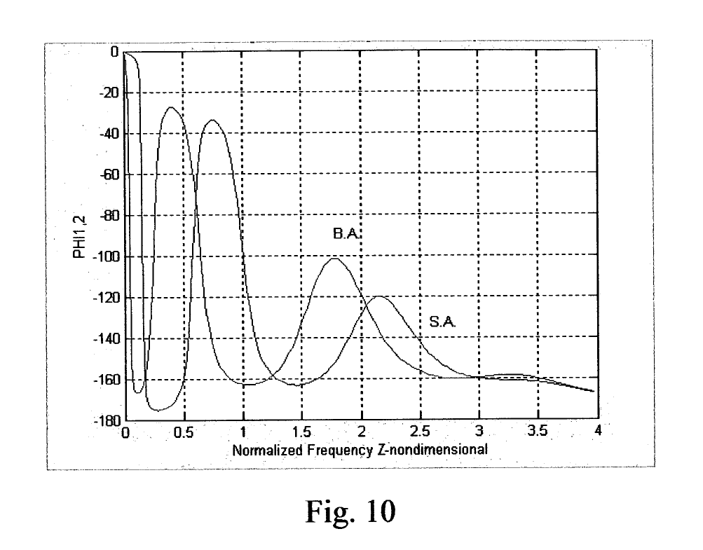

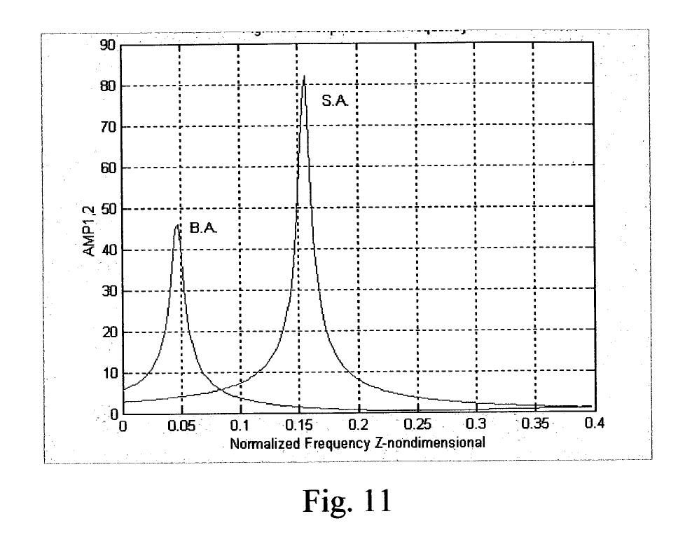

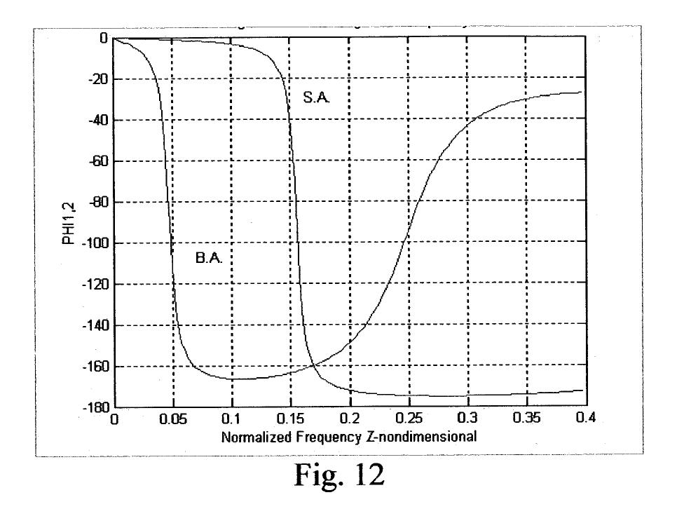

FIG. 9 shows the normalized amplitude as a function of a normalized frequency, and FIG. 10 shows the phase angle as a function of the normalized frequency. The normalized amplitude and the phase angle for low normalized frequencies are shown in FIGS. 11 and 12, respectively.

As can be understood from FIGS. 5-12, the response to excitation at the external point of contact is sensitive to the mechanical properties of the tissue deep inside the body. Hence, mechanical properties of internal tissues are characterized by external measurements.

### Example 2

### A Two Dimensional Model for a Peripheral Vascular Case

The present example is a two dimensional model which simulates a continuous mass system of an artery, a plaque (if exist in the artery) and the adjacent skin. The model comprises a system of a plurality of particles each particle has two degrees-of-freedom. Thus, a system of M particles has N=2M degrees-of-freedom.

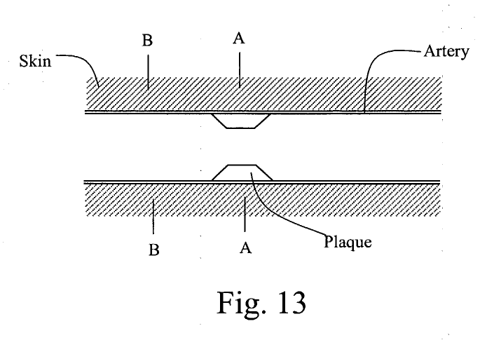

Reference is now made to FIG. 13, showing an artery carrying a plaque which is located on the wall of the artery. The artery is below the skin of the subject which is shown as a gray area in FIG. 13. The two dimensional model below simulates the artery along a perpendicular cross section designated “A-A” in of FIG. 13.

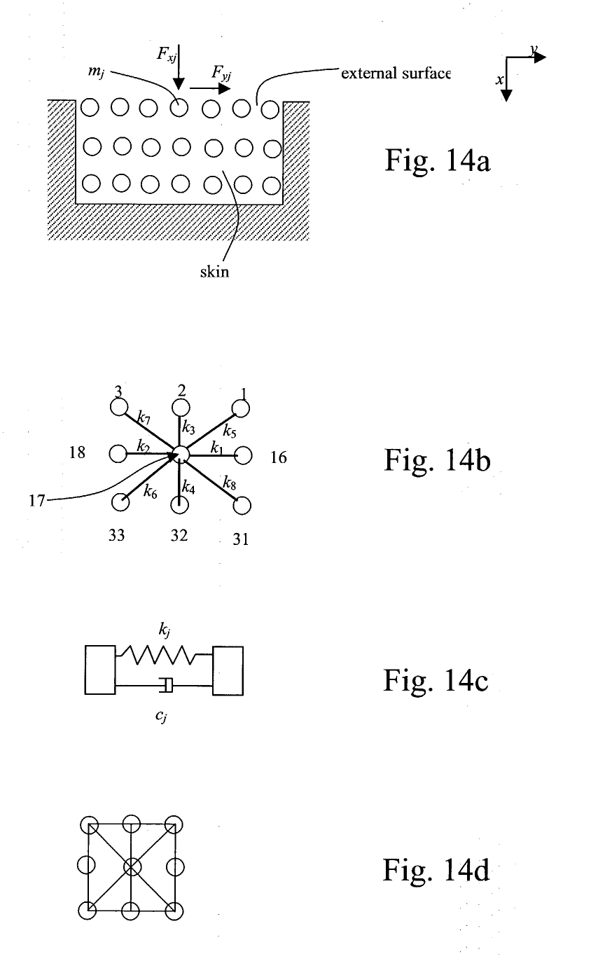

FIGS. 14a-d are an illustration of the two dimensional model which consists of a plurality of particles. FIG. 14a shows the particles, each represented as a circle in FIG. 14a. FIG. 14b shows coupling of a certain particle designated 17, with its eight neighbours, designated 1, 2, 3, 16, 18, 31, 32 and 33. FIG. 14c illustrates the forces between two neighboring particles. As detailed in Example 1, two mutual forces are between the particles, the elastic force, represented by a spring and the dissipative force, represented by a viscous damper. Each of the eight neighbors of particle 17 (see FIG. 14b) applies a different force onto particle 17. These are represented by eight different spring constants kᵢ, i=1, . . . , 8. There are four inclined spring constants, k₅, k₆, k₇ and k₈, which simulate a real matter having a non-zero Poisson's ratio. FIG. 14d shows a square region of particles, which simulates the artery. Shown in FIG. 14d a 3×3 region of particles, however larger regions may be considered as well.

A displacement of the jth particle in a direction normal to the external surface is denoted in FIG. 14a by xⱼ(t) and a displacement of a particle j in a tangential direction to the external surface is denoted by yⱼ(t). A driving force is applied to an external particle j, positioned on the external surface. The components of the driving force are shown as arrows in FIG. 14a and denoted Fₓⱼ and F_{yj} for the x and y direction, respectively. The driving force of the present example is given by the equation:

Fⱼ(t)=F₀ⱼ sin(ωt), (EQ. 5)

where ω is a circular frequency and F₀ⱼ (constant) force amplitude. In practice, constant force amplitude may be achieved using an MLD having a very soft spring.

Referring again to FIG. 14b, the motion of particle mass 17 is described by two linear differential equations of motion (one for each degree-of-freedom of the particle). One of ordinarily skill in the art would appreciate that without a driving force these equations are:

$$\begin{matrix}{m}_{17} {x}_{17} = - {k}_{1} ( {x}_{17} - {x}_{16} ) - {k}_{2} ( {x}_{17} - {x}_{18} ) - \\ {k}_{5} ( {y}_{17} + {x}_{17} - {y}_{1} - {x}_{1} ) / 2 - {k}_{6} ( {y}_{17} + {x}_{17} - {y}_{33} - {x}_{33} ) / 2 + \\ {k}_{7} ( {y}_{17} - {x}_{17} - {y}_{3} + {x}_{3} ) / 2 + {k}_{8} ( {y}_{17} - {x}_{17} - {y}_{31} + {x}_{31} ) / 2\end{matrix} \\ \begin{matrix}{m}_{17} {y}_{17} = - {k}_{3} ( {y}_{17} - {y}_{2} ) - {k}_{4} ( {y}_{17} - {y}_{32} ) - \\ {k}_{5} ( {y}_{17} + {x}_{17} - {y}_{1} - {x}_{1} ) / 2 - {k}_{6} ( {y}_{17} + {x}_{17} - {y}_{33} - {x}_{33} ) / 2 - \\ {k}_{7} ( {y}_{17} - {x}_{17} - {y}_{3} + {x}_{3} ) / 2 - {k}_{8} ( {y}_{17} - {x}_{17} - {y}_{31} + {x}_{31} ) / 2\end{matrix} \qquad ( EQ . 6 )$$

To find the steady forced vibrations of a particle (say, the jth particle) which is subjected to the driving force of Equation 5, one needs to add the driving force to the right hand side of the corresponding equation. A particular solution to the resulting equation is of the form xₗ(t)=x₀ₗe^{iωt}, where xₗ, x₀ₗ are complex numbers. Substituting the solution to the differential equation one obtains a system of N×N linear equations with constant coefficients, which can be written in the following matrix form:

[aᵢⱼ(ω,par.)]{Q₀ⱼ}={F₀ⱼ}, (EQ. 7)

where Q₀ⱼ, representing the amplitudes x₀ⱼ or y₀ⱼ, and par stands for the lumped parameters of the model (elasticity, mass, viscosity). The solution of Equations 7 depends on the frequency of the driving force, ω. As stated, the observable is the particle which is in contact with the MLD, i.e., the particle onto which the driving force is applied.

For a symmetrical system with regards to the center in which the force is applied at the center of the artery (at position A, see FIG. 13) y₀ⱼ is decoupled from X₀ⱼ. In an asymmetric system, there is a dynamic coupling between the perpendicular and tangential displacements, xⱼ and yⱼ. In other words, each component of the driving force excites both xⱼ and yⱼ.

It is expected that a change in the parameters of the system (e.g., different masses and/or different spring constants) would result in different responses. Thus, benign or malignant regions of the artery inside the skin are expressed by different parameters, thereby leading to different responses to a given driving frequency. These differences allow identification of the type of plaque. Specifically, hard and dense plaque, which is less dangerous, is expressed by heavy mass particles and hard springs, while soft and light plaque, which is highly dangerous, is expressed by light mass particles and soft springs.

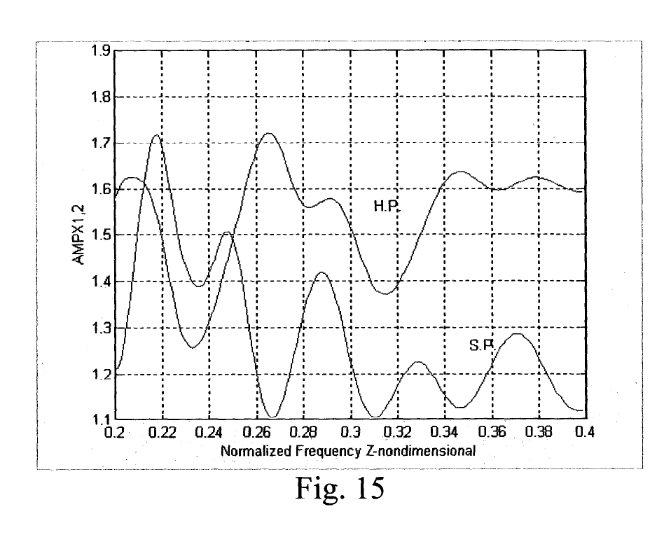

A representative system comprising 451 particles (a 11×41 matrix) was analyzed and the results of the frequencies responses are described below with references to FIGS. 15-22.

Curves on FIGS. 15-22 which are designated by the letters SP correspond to calculations using a set of parameters which is selected to simulate soft plaque, curves which are designated by the letters HP correspond to calculations using a set of parameters which is selected to simulate hard plaque, and curves which are designated by the letters CP correspond to calculations using a set of parameters which is selected to simulate clean or benign artery.

FIG. 15 shows a normalized amplitude, AMPXᵢ, as a function of the normalized frequency, Z, for excitation of hard plaque and soft plaque in x direction. The normalized frequency is defined above (see Equation 4) and AMPXᵢ, is defined as:

$${AMPX}_{i} = \sqrt{{Rx}_{i}^{2} + {Ix}_{i}^{2}} \times ( \frac{k}{{F}_{{0}_{{x}_{i}}}} ) , \qquad ( EQ . 7 )$$

where Rxᵢ=Real(x₀ᵢ) and Ixᵢ=Imaginery(x₀ᵢ).

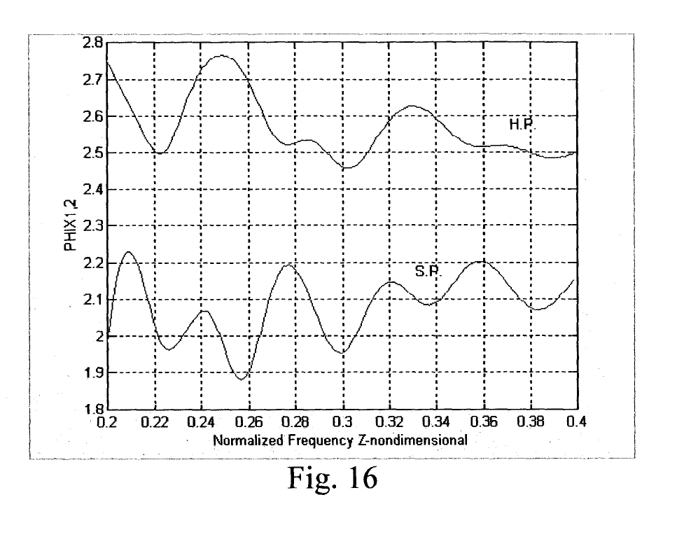

FIG. 16 shows a phase angle, PHIXᵢ, as a function of Z, again, for excitation of hard plaque and soft plaque in x direction. PHIXᵢ, is defined as:

PHIXᵢ=tan⁻¹(Ixᵢ/Rxᵢ). (EQ. 8)

As can be seen from FIG. 15 and FIG. 16, the differences in responses between hard plaque and soft plaque are considerable for excitation in x direction.

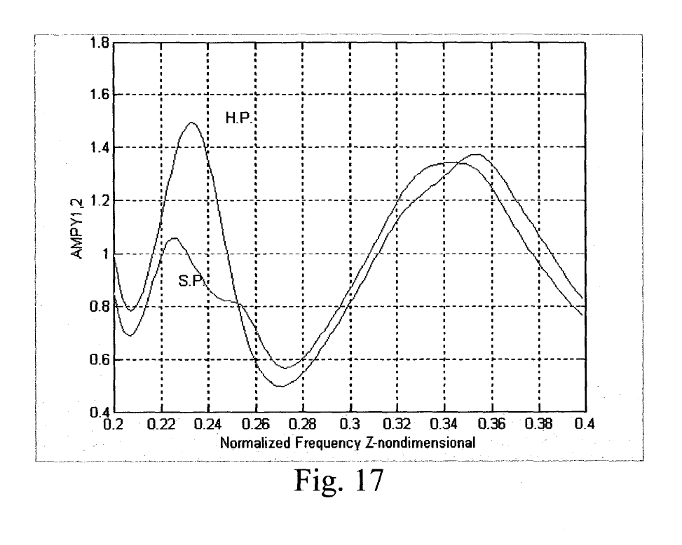

FIG. 17 shows a normalized amplitude, AMPYᵢ, as a function of Z, for excitation of hard plaque and soft plaque in y direction. Similarly to Equation 7, the definition of AMPYᵢ is:

$${AMPY}_{i} = \sqrt{{Ry}_{i}^{2} + {Iy}_{i}^{2}} \times ( \frac{k}{{F}_{{0}_{{y}_{i}}}} ) , \qquad ( EQ . 9 )$$

where Ryᵢ=Real(y₀ᵢ) and Iyᵢ=Imaginery(y₀ᵢ).

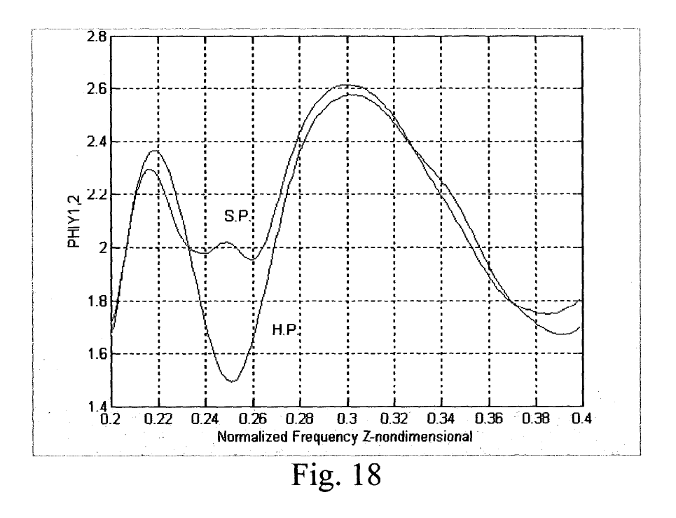

FIG. 18 shows a phase angle, PHIYᵢ, as a function of Z, again, for excitation of hard plaque and soft plaque in y direction. PHIYᵢ, is defined as:

PHIYᵢ=tan⁻¹(Iyᵢ/Ryᵢ). (EQ. 10)

As can be seen from FIG. 17 and FIG. 18, the differences in responses between hard plaque and soft plaque are less considerable for excitation in y direction than for excitation in x direction. Nevertheless, the responses of hard plaque and soft plaque differ.

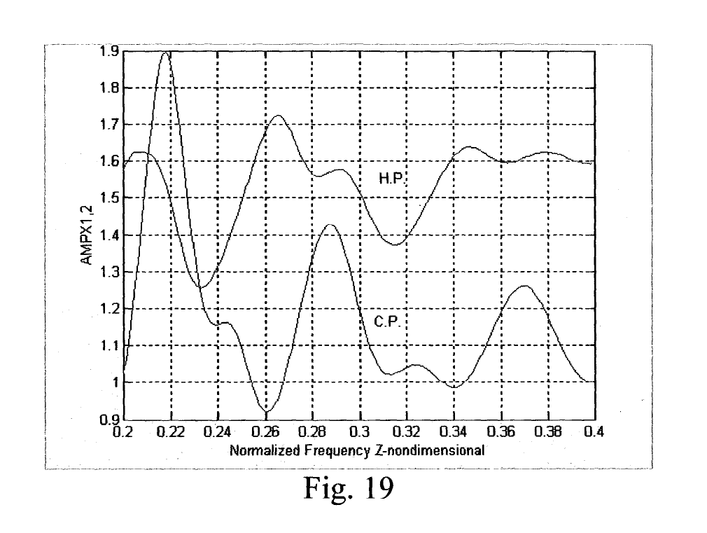

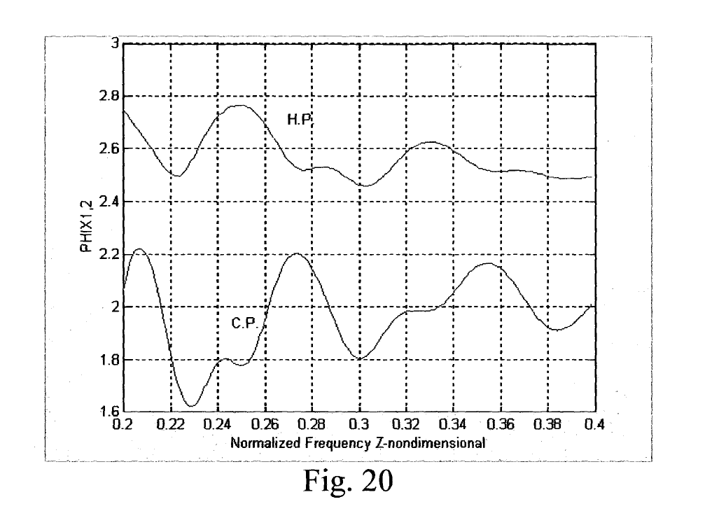

Comparison between hard plaque and benign clean artery for excitation in x direction are shown in FIGS. 19-20, where FIG. 19 shows AMPXᵢ and FIG. 20 shows PHIXᵢ. As can be seen, there is a significant difference between the responses of hard plaque and benign clean artery.

The position in which the driving force is applied reflects on the frequency response spectrum as well. This may be simulated by selecting a different particle of the system to be excited, e.g., by selecting a particle located at a perpendicular cross section designated “B-B” in of FIG. 13.

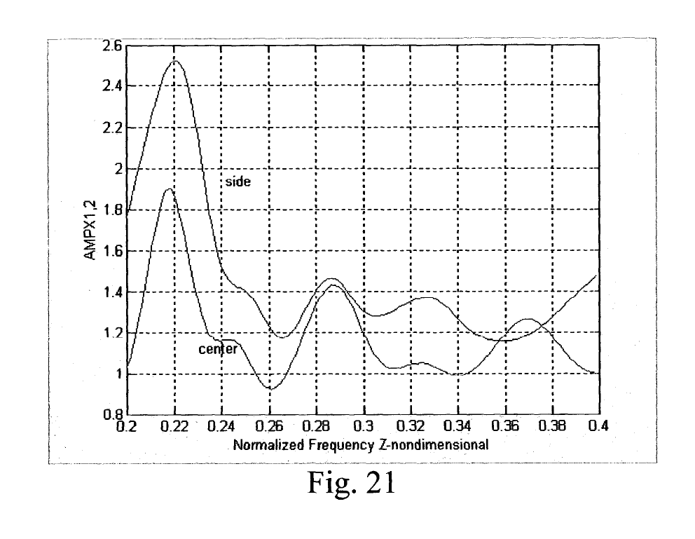

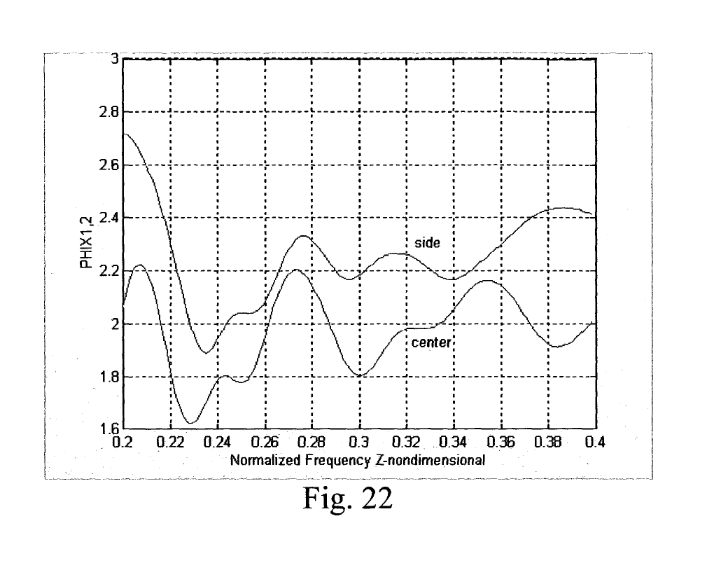

FIGS. 21-22 show a comparison between different positions of the excited particle relative to the position of the clean artery. The corresponding curves are labeled by “center” for central excitation over the artery and “side” for off-central excitation off the artery.

FIG. 21 shows AMPXᵢ as a function of Z and FIG. 22 shows PHIXᵢ, as a function of Z, for center and side excitations of a benign clean artery. As can be seen, responses depend on the position in which the force is applied, hence, responses can serve for determining the location of an artery.

### Example 3

### A Two Dimensional Model for a Dermal or Sub-Dermal Case

The present example is of a two dimensional model which simulates a continuous mass system of a dermal or sub-dermal lesion surrounded by benign skin tissues. The model comprises a system of a plurality of particles each particle has two degrees-of-freedom. The interactions between the particles and the applied driving force are as in Example 2 and therefore governed by the same set of equations.

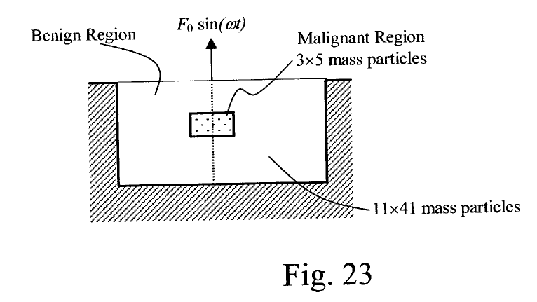

Reference is now made to FIG. 23, showing a portion of a suspected region of a skin. The benign region is shown as a bright area in FIG. 23 and the lesion to be characterized is shown as a dotted area within the bright area.

The mechanical properties of a dermal or sub-dermal lesion differ significantly from a benign skin tissue: the former is known to be much softer than the latter. In this example the suspected region of a skin was simulated by a system comprising 451 particles (a 11×41 matrix), the parameters of 15 of which (a 3×5 matrix) were selected in accordance with a malignant lesion characteristics (small masses and spring constants), and the parameters of all other particles were selected in accordance with a benign skin tissue characteristics. The ratio between the parameters of the malignant lesion to the parameters of benign skin tissue was 1:2, respectively.

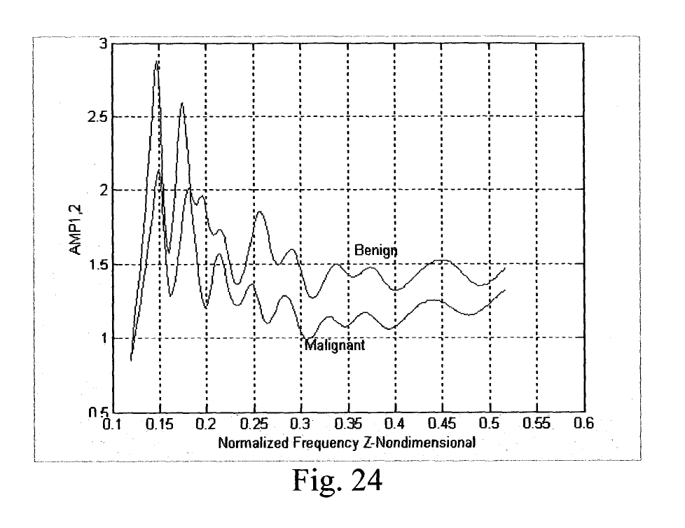

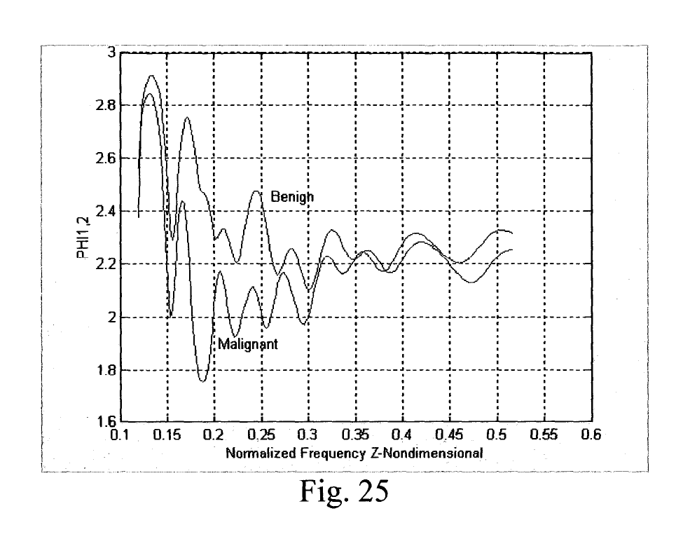

FIG. 24 shows AMPXⱼ as a function of Z for excitation of benign skin tissue and malignant lesion in x direction. FIG. 25 shows PHIXⱼ as a function of Z for excitation of benign skin tissue and malignant lesion in x direction.

For both amplitude and phase angle a significant difference between the responses of benign and malignant tissues was observed, as shown in FIG. 24 and FIG. 25, respectively.

### Example 4

### Decalcification of Bones

At the preliminary stage of bone's decalcification the density of the bone remains unchanged while the elasticity is known to decrease by about 30%.

In this example a bone is modeled by continuous mass beam at a transverse vibration mode. The natural frequency of the nth mode, ωₙ, of a beam is well known, and is given by:

ωₙ=Aₙ\sqrt{E I/Aρl⁴}, (EQ. 11)

where E is Young's Modulus, I is an area moment of inertia, A is an area of the cross section of the beam, ρ is a density of the beam, l is a length of the beam and Aₙ, n=1,2, . . . are constants that depend on the boundary conditions.

As a consequence to bone's decalcification the natural frequency, ωₙ, decreases by about 16%. T hus, bone's decalcification affects the frequency response, which effect is measurable as exemplified in the previous examples.

### Example 5

### A Design of the Mechanical Linkage Device (MLD)

As stated hereinabove, in one embodiment the MLD is used at a specific position both to apply the force and to measure the displacement with minimal distortions. The dynamical interaction between the MLD and the tested improves the capability to distinguish between different biological materials inside the body.

Ideally, an optimal MLD would be a very soft and very light spring, positioned between a vibrating table and the body, where the vibration amplitude of the vibrating table is much larger than the vibration amplitude of the point of contact with the body. In addition, the natural frequency of the spring of an ideal MLD is much higher than the forcing frequency so as to prevent dynamical distortion. Practically, however, such MLD is rarely attainable.

This example demonstrates an MLD design which is sufficient to provide the desired functionality of the MLD, namely, the capability to apply the force and to measure the displacement with minimal distortions, and an enhanced capability to distinguish between different biological materials.

In this example, the spring is realized as a continuous mass flexible member having many natural frequencies and vibration modes. One ordinarily skilled in the art would appreciate that, if the measured quantity is the ratio between the motion characteristics of the body to the motion characteristics of the vibrating table, such multiplicity of frequencies of the driving force does not interfere with the objects of the present embodiment.

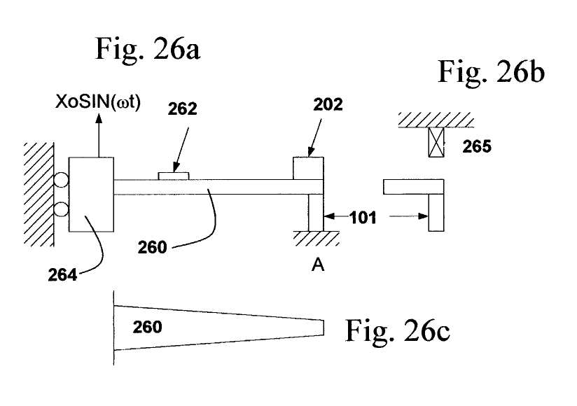

FIGS. 26a-c illustrates the MLD of this example. The MLD comprises a thin variable width beam spring 260 which is connected to contact tip 101 on one end and to a vibrating table 264 on the other end. Contact tip 101 touches the body at a point designated in FIG. 26a by A.

For the purpose of measuring the vibration's displacement the MLD comprises a strain gage 262 and/or a proximity sensor 265. The use of strain gage and/or proximity allows the measurement of the displacement without addition of mass to the MLD. Strain gage 262 also measures the preload which is needed to be measured and controlled because of the nonlinearity of the biological materials which affect the response.

Senor 202 is a piezoelectric micro mechanical sensor which is simple and practical. Nevertheless, the mass of sensor 202, despite being small (about 0.5 gr.) decreases the natural frequency of the spring.

The dynamical response of point A was simulated considering a system consisting of the body and the MLD. The simulation results in the following set of linear equations:

|aᵢⱼ|{yⱼ}={fᵢ}, (EQ 12)

where aᵢⱼ are the elements of a square matrix, yⱼ is the displacements vector, and fⱼ is the force vector due to the vibrator motion, all of which depend on the input frequency. The required output from Equation 12 is y_{A}, the displacement of the contact point A.

The design of the MLD includes optimization of the input frequency, the overall size and the natural frequency of the MLD. Small size MLD (compared to the local parts of the body) corresponds to higher sensitivity; higher natural frequency corresponds to substantial constant force excitation. Hence, judicious choice of the parameters results in the desired dynamical interaction between the body and the MLD, which increases the sensitivity to the mechanical properties inside the body.

### Example 6

### Tissue Sorting According to Elasticity

In this example, tissues were modeled by a man made structural model which was used to verify the ability to characterize tissues according to their elasticity.

Method

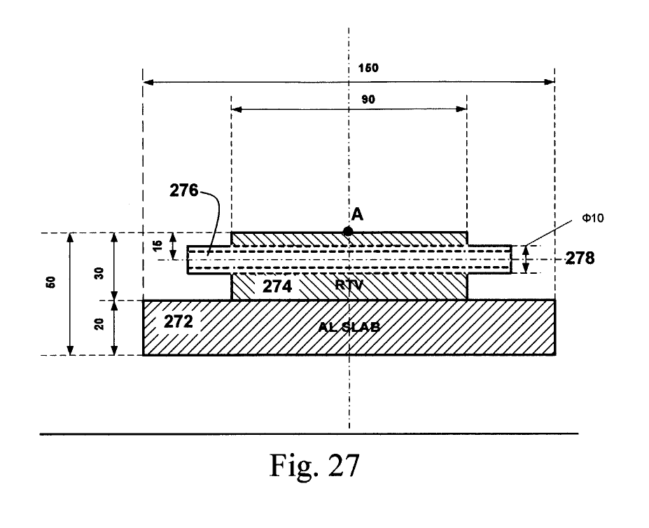

FIG. 27 shows the experimental setup for simulating the tissue. The structural model included an aluminum square plate 272, 20 mm in thickness and 150 mm in width, which was used as a base. Plate 272 was concentrically covered by a square slab 274 made of soft silicone rubber (RTV-410), 30 mm in thickness and 90 mm in width. A latex tube 276, 10 mm in diameter, was introduced into the volume of slab 274, so that the central axis 278 of tube 276 was 15 mm below the top of slab 274. The purpose of tube 276 was to facilitate replacements of test inserts, as further detailed below. All the parts of the structural model were strongly cemented to one another.

The complex frequency response of the model at a point of contact, at the center of slab 274 with the body was measured using the devices and methods of the present invention as further detailed hereinabove.

Results

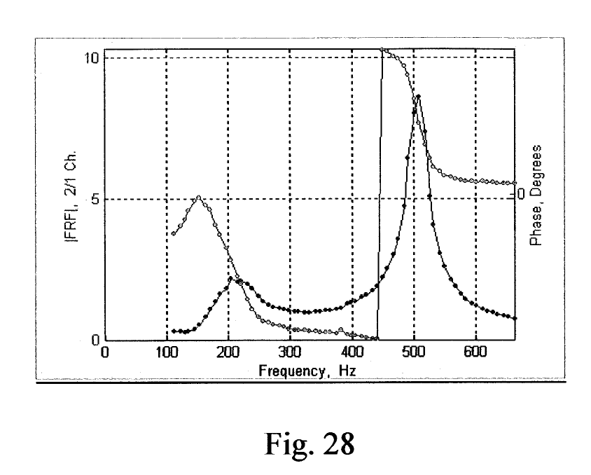

FIG. 28 shows the absolute value and the phase of the frequency response as a function of the frequency. The salient features of this frequency response come from the nature of the tissue and the frequency range chosen. As shown in FIG. 28, there are two resonance frequencies at a range of 100-700 Hz where the upper frequency resonance (at about 510 Hz) has a larger absolute value and a steeper shape than the lower frequency resonance (at about 220 Hz).

This response may be further analyzed by comparing the responses of various contact points along a scanning path. In medical application, for example, the operator may select a geometrical path to follow (a line, a circle, a curve or any other open or closed path). The desired resolution of the examination dictates the number and density of points at which the response is to be measured. The measured frequency responses of the tissue at the various points are recorded and used for the characterization of the tissue.

In this experiment, two geometrical scans were performed, using two test inserts, along and above axis 278 of tube 276 with a resolution of 1 mm space between two adjacent reading-points. The first insert into the tube was a copper rod, 10 mm in length, and the second insert was a rubber plug, 20 mm in length, both positioned so as to touch the inner wall of the tube.

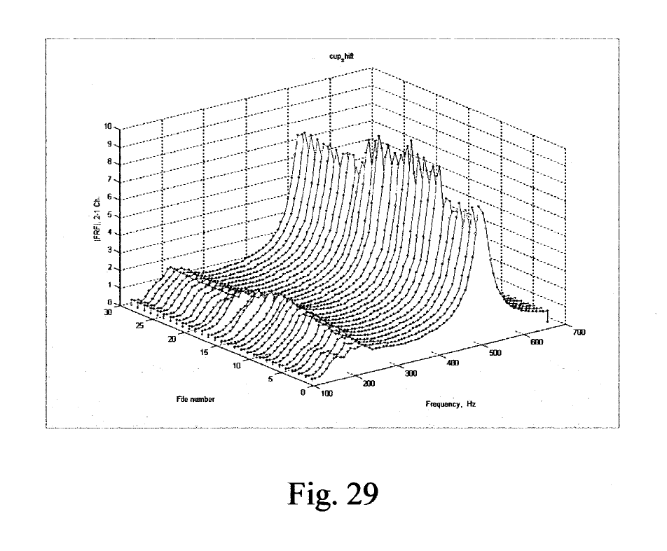

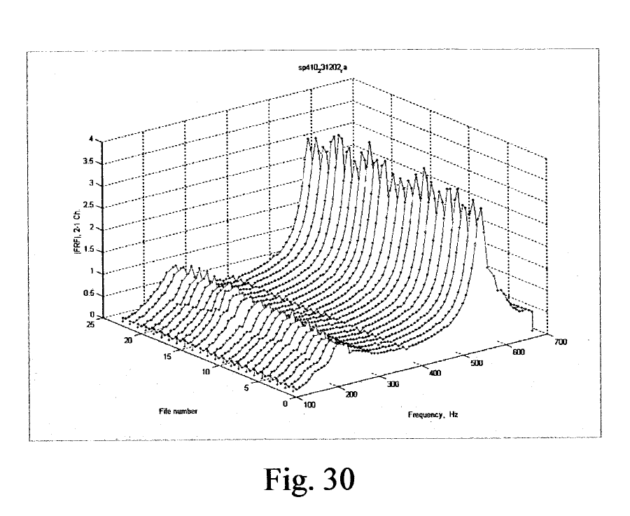

The results of all the responses acquired are displayed as three dimensional “waterfall” plots in FIG. 29 (copper insert) and FIG. 30 (rubber insert). The three axes FIGS. 29-30 are the frequency, the scan distance and the amplitude.

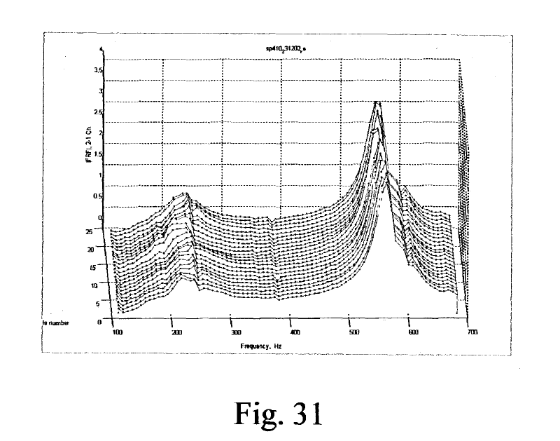

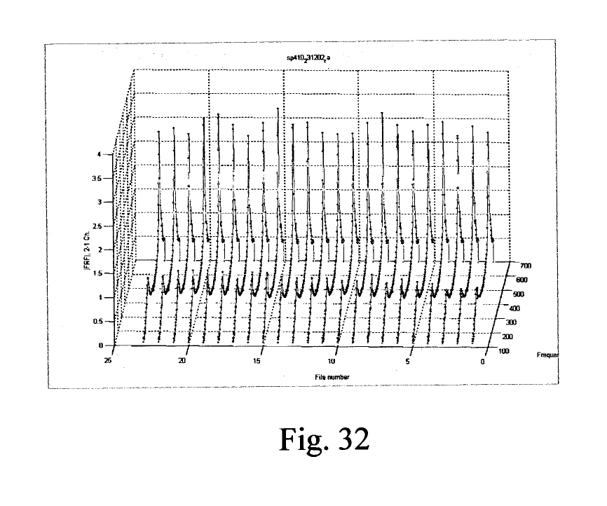

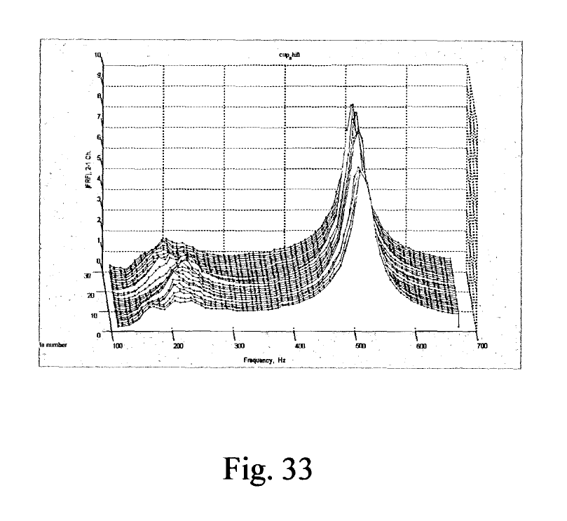

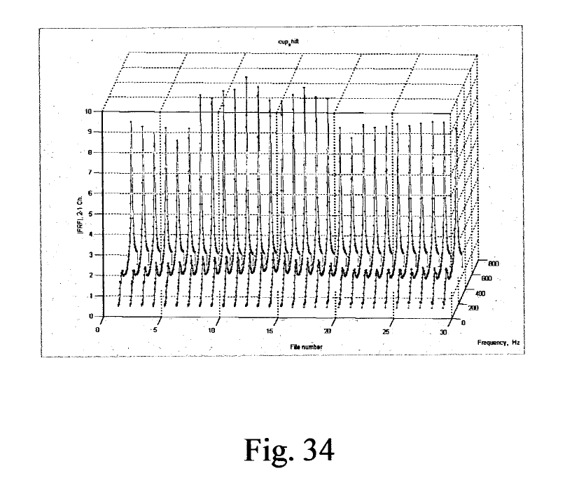

FIGS. 31-34, show projections the “waterfall” plots of FIG. 29-30 onto the frequency-amplitude plane (FIG. 31 for a rubber insert and FIG. 33 for a copper insert) and the distance-amplitude plane (FIG. 32 for rubber and FIG. 34 for copper). As can be seen from FIGS. 31-34, the copper insert shows upshift of the lower frequency resonance, while the rubber insert shows a downshift of the lower resonance. All the scanned points on the tube have a similar two-resonance behavior, where at each point above the insert (disturbed region) the resonance frequency is shifted and the absolute value and phase change. On would appreciate the good correlations between the insert length and location in the tube and the changes of elastic behavior of the system on the distance axis.

Data Analysis

Following is a detailed description of a method used to quantify and sort the elastic behavior changes along the trace of a scan based on the above findings.

First, the values of the maxima of the resonance peaks in all the scanned geometrical points were identified. Each two resonance frequencies corresponded to two values: (i) for an undisturbed tube region; and (ii) for a disturbed tube region (with an insert). Averaging taken on the frequencies in these two regions catered for small structural variations.

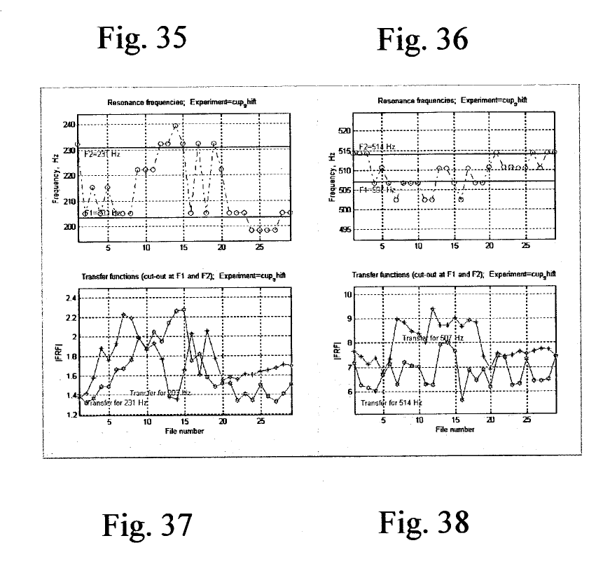

FIGS. 35 and 36 show, for a copper insert case, the resonance frequency shifts for the lower (FIG. 35) and upper (FIG. 36) frequency resonances. The resonance frequency plot as a function of the geometrical location on the disturbed setup has an approximately rectangular shaped deviation at the copper insert region. The size and sign of this deviation are determined by the elasticity and geometry of the complete setup. Similar plots were obtained for the undisturbed setup.

Second, the absolute value of the frequency response function at each of the averaged frequency maxima was plotted. Thus, plots obtained for the lower frequency resonance and for the upper frequency resonance.

FIG. 37 shows the plot obtained for copper insert at the frequency of both the disturbed and the undisturbed regions at the lower resonance range. This plot contains two curves. One curve is at the averaged unshifted frequency, and its disturbed region is identified by a valley. The valley may exhibit either a rectangular or rounded shape where the insert is in the tube. The other curve is at the averaged shifted frequency, and its disturbed region is identified by a bulge. The bulge may exhibit either a rectangular or rounded shape where the insert is in the tube.

FIG. 38 shows the plot obtained at the frequency of the both the disturbed and the undisturbed regions at the higher resonance range. This plot contains two curves. One curve is at the averaged unshifted frequency, and its disturbed region is identified by a valley. The valley may exhibit either a rectangular or rounded shape where the insert is in the tube. The other curve is at the averaged shifted frequency, and its disturbed region is identified by a bulge. The bulge may exhibit either a rectangular or rounded shape where the insert is in the tube.

Whenever the shifted frequency is higher than the unshifted frequency, the insert makes the complete setup stiffer. The experimental results show that copper is stiffer than rubber plate at the low resonance frequency and softer at the high resonance frequency.

A similar procedure was used on the phase of the frequency response function.

Third, the values of absolute values on the above plots were averaged, both in and out of the disturbed region. On each of the plots, the ratio between the absolute value of the disturbed and the absolute value of the undisturbed frequency range is indicative to the elasticity and geometry of the system.

The results of the copper insert are given in Table 1, below. In Table 1, fₗ, and fₕ are, respectively, the low and high resonances; Δfₗ and Δfₕ are, respectively, the frequency shifts at the disturbed region in these two resonances; R₁ and R₁′ are, respectively, the ratios of the absolute value of frequency response function in the unshifted and shifted low resonance frequency; and R₂ and R₂′ are, respectively the equivalent values at the high resonance frequency.

By simple logic, if the insert is stiffer than the rubber plate, then Δf>0, R_{1,2}<1 and R′_{1,2}>1, whereas if the insert is softer than the rubber plate, then Δf<0, R_{1,2}>1 and R′_{1,2}<1. Thus, the values of R_{1,2}, R′^{1,2}, and Δf are characteristics of the stiffness (or softness) of the insert and may be used to define the degree of stiffness of the insert, and later of the plaque.

TABLE 1

| Test | Low Frequency Peak, f₁ = 207 Hz | | | High Frequency Peak, fₕ = 514 Hz | | |
|---|---|---|---|---|---|---|
| Insert | Frequency | Level Ratio | | Frequency | Level Ratio | |
| Material | Shift Δf₁ | R₁ | R₁′ | Shift Δfₕ | Rₕ | Rₕ′ |
| Copper | 24 Hz | 1.63 | 0.61 | 7 Hz | 1.5 | (noisy) |

Table 1 demonstrates that the copper insert in rubber plate is characterized by different Δf at different resonance frequencies. Specifically, the copper is characterized by R₁, R₁′ at the low frequency peak region and Rₕ, Rₕ′ at the high frequency peak region.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of characterizing a tissue present in a predetermined location of a body of a subject, the method comprising:
generating mechanical vibrations at a position adjacent to the predetermined location, said mechanical vibrations are at a frequency ranging from 10 Hz to 10 kHz;
scanning said frequency of said mechanical vibrations; and
measuring a frequency response spectrum from the predetermined location, thereby characterizing the tissue.

2. The method of claim 1, wherein the tissue forms a part of an organ.

3. The method of claim 1, wherein the tissue forms a part of an internal organ.

4. The method of claim 1, wherein the tissue forms a portion of a tumor.

5. The method of claim 1, wherein the tissue forms a portion of an internal tumor.

6. The method of claim 1, wherein the tissue is a pathological tissue.

7. The method of claim 1, wherein the tissue forms a part of, or is associated with, a blood vessel tissue.

8. The method of claim 7, wherein said blood vessel tissue is selected from the group consisting of a blood clot, an occlusive plaque and a vulnerable plaque.

9. The method of claim 1, wherein the tissue forms a portion of a bone.

10. The method of claim 1, wherein the tissue is a stenotic tissue.

11. The method of claim 1, wherein said measuring said frequency response spectrum comprises measuring an amplitude as a function of said frequency.

12. The method of claim 1, wherein said measuring said frequency response spectrum comprises measuring a phase angle as a function of said frequency.

13. The method of claim 1, further comprising calculating at least one mechanical property of the tissue from said frequency response spectrum.

14. The method of claim 13, wherein said mechanical property is an elastic constant.

15. The method of claim 13, wherein said mechanical property is selected from the group consisting of an elastic modulus, a Poisson's ratio, a shear modulus, a bulk modulus and a first Lamé coefficient.

16. The method of claim 1, wherein said position is on a skin of the body.

17. The method of claim 1, wherein said position is close to a blood vessel-of-interest.

18. The method of claim 17, wherein said blood vessel-of-interest is selected from the group consisting of a carotid, a femoral vessel and an abdominal aorta.

19. The method of claim 1, wherein said position is close to a lesion selected from the group consisting of a dermal lesion, a sub-dermal lesion and an internal lesion.

20. The method of claim 1, wherein said position is close to a bone.

21. The method of claim 1, wherein said position is close to a thorax.

22. The method of claim 1, wherein said mechanical vibrations are perpendicular to the body.

23. The method of claim 1, further comprising endoscopically inserting an endoscopic device having an imaging device into the subject, and using said imaging device for imaging the subject so as to determine a position of the tissue.

24. The method of claim 23, wherein said generating said mechanical vibrations is performed within the subject by said endoscopic device.

25. The method of claim 23, wherein said imaging device is selected from the group consisting of an intra vascular ultra sound device, an intra vascular magnetic resonance device and a camera.

26. The method of claim 1, wherein said generating said mechanical vibrations is performed such that said mechanical vibrations are inclined to the body, by a predetermined inclination angle.

27. The method of claim 26, wherein said predetermined inclination angle is selected so as to enhance data acquisition.

28. The method of claim 26, wherein said step of generating mechanical vibrations is repeated a plurality of times, each time with a different inclination angle.

29. The method of claim 1, wherein said step of generating mechanical vibrations is repeated a plurality of times, each time in a different location.

30. The method of claim 1, wherein said frequency of said mechanical vibrations is selected from the group consisting of a single frequency, a superposition of a plurality of frequencies, a continuous frequency scan (chirp), and a band-limited white noise frequency.

31. The method of claim 1, wherein said generating said mechanical vibrations is by a mechanical vibrations generating assembly.

32. The method of claim 1, wherein said mechanical vibrations generating assembly is constructed and designed so as to minimize effects of environmental noise.

33. The method of claim 31, wherein said mechanical vibrations generating assembly comprises a at least one mechanical linkage device for transferring said mechanical vibrations to the body.

34. The method of claim 33, wherein at least one of a size and a natural frequency of said at least one mechanical linkage device is selected so as to increase dynamical interactions between the tissue and said at least one mechanical linkage device.

35. The method of claim 33, wherein said at least one mechanical linkage device is characterized by a plurality of natural frequencies, and further wherein at least one frequency of said plurality of natural frequencies is higher than said frequency of said mechanical vibrations.

36. The method of claim 33, wherein said at least one mechanical linkage device comprises a variable width beam spring.

37. The method of claim 33, wherein said at least one mechanical linkage device comprises a strain gage for measuring displacement of said at least one mechanical linkage device.

38. The method of claim 33, wherein said at least one mechanical linkage device comprises a proximity sensor for measuring displacement of said at least one mechanical linkage device.

39. The method of claim 1, wherein said generating said mechanical vibrations is by transmitting mechanical vibration from a first mechanical linkage device to a second mechanical linkage device via at least one mechanical sensor.

40. The method of claim 39, wherein said first and said second mechanical linkage devices are each independently membranes.

41. The method of claim 40, wherein said membranes are made of a material selected from the group consisting of a plastic and a metal.

42. The method of claim 40, wherein said membranes are piezo-polymeric membranes.

43. The method of claim 32, wherein said mechanical vibrations generating transducer assembly comprises a tubular transducer.

44. The method of claim 31, wherein said mechanical vibrations generating assembly comprises at least one contact-tip.

45. The method of claim 44, further comprising bulging said at least one contact-tip out of an encapsulation of said mechanical vibrations generating assembly so as to touch the tissue.

46. The method of claim 44, wherein said at least one contact-tip comprises a plurality of contact-tips arranged in a matrix-like arrangement.

47. The method of claim 44, wherein said at least one contact-tip is sterilizable.

48. The method of claim 44, wherein said at least one contact-tip comprises at least one sterilizable cover.

49. The method of claim 44, wherein said at least one contact-tip is disposable.

50. The method of claim 31, wherein said mechanical vibrations generating assembly comprises a mechanical vibrations generating transducer assembly, said mechanical vibrations generating transducer assembly is operable to convert electrical signals into mechanical motions.

51. The method of claim 50, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly and an electrostatic mechanical vibrations generating transducer assembly.

52. The method of claim 31, wherein said mechanical vibrations generating assembly comprises at least one mechanical sensor.

53. The method of claim 52, wherein said at least one mechanical sensor is selected from the group consisting of a contact sensor and a remote sensor.

54. The method of claim 52, wherein said at least one mechanical sensor is selected from the group consisting of an acceleration sensor, a force sensor, a pressure sensor and a displacement sensor.

55. The method of claim 31, wherein said mechanical vibrations generating assembly comprises a mechanism for isolating said mechanical vibrations generating assembly from environmental vibrations.

56. The method of claim 55, wherein said mechanism is operable to independently move in three orthogonal directions.

57. The method of claim 55, wherein said mechanism is operable to independently rotate in at least two orthogonal directions.

58. The method of claim 31, further comprising transmitting an electrical signal to said mechanical vibrations generating assembly.

59. The method of claim 31, wherein said measuring is by receiving an electrical signal transmitted from said mechanical vibrations generating assembly.

60. The method of claim 59, further comprising displaying said electrical signal transmitted from said mechanical vibrations generating assembly on a display.

61. The method of claim 60, wherein said display is selected from the group consisting of an oscilloscope, a spectrum analyzer, a processor display and a printer.

62. The method of claim 1, further comprising classifying said frequency response spectrum.

63. The method of claim 62, wherein said classifying said frequency response spectrum comprises:
(a) identifying resonance peak maxima of said frequency response spectrum;
(b) from said resonance peak maxima, determining a first type of maximum being indicative of a first type of tissue, and a second type of maximum being indicative of a second type of tissue; and
(c) using said first type of maximum and said second type of maximum to classify said first and said types of tissue.

64. The method of claim 63, wherein said step (c) comprises calculating a ratio between said first type of maximum and said second type of maximum.

65. The method of claim 63, further comprising averaging said resonance peak maxima.

66. The method of claim 63, wherein said first and said second types of maxima are determined by absolute values of said resonance peak maxima.

67. The method of claim 63, wherein said first and said second types of maxima are determined by shapes of said resonance peak maxima.

68. The method of claim 63, wherein said first and said second types of maxima are determined by frequency shifts of said resonance peak maxima.

69. The method of claim 62, wherein said classifying comprises:
(a) constructing a physical model of a plurality of harmonic oscillators, said physical model comprises a set of parameters and being characterized by a plurality of equations of motion;
(b) simultaneously solving said plurality of equations of motion so as to provide at least one frequency response; and
(c) comparing said at least one frequency response with said frequency response spectrum;
thereby classifying said frequency response spectrum.

70. The method of claim 69, wherein said physical model is an N degree-of-freedom physical model, said N is a positive integer.

71. The method of claim 69, wherein said plurality of harmonic oscillators are coupled harmonic oscillators.

72. The method of claim 69, wherein at least a portion of said plurality of harmonic oscillators are damped harmonic oscillators.

73. The method of claim 69, wherein at least a portion of said plurality of harmonic oscillators are forced harmonic oscillators.

74. The method of claim 69, wherein said set of parameters comprises at least one constant of inertia and at least one elastic constant.

75. The method of claim 74, wherein said constant of inertia is mass and further wherein said elastic constant is a spring constant.

76. The method of claim 74, wherein said constant of inertia is inductance and further wherein said elastic constant is a reciprocal of capacitance.

77. The method of claim 69, further comprising repeating said steps (a)-(c) at least once, each time using different set of parameters.

78. The method of claim 69, wherein said set of parameters represent dynamic stiffness and density of the structural material.

79. A system for characterizing a tissue present in a predetermined location of a body of a subject, the system comprising:
a mechanical vibrations generating assembly for generating mechanical vibrations at a position adjacent to the predetermined location, said mechanical vibrations are at a frequency ranging from 10 Hz to 10 kHz; and a control unit for scanning said frequency of said mechanical vibrations, and for measuring a frequency response spectrum from the predetermined location, thereby to characterize the tissue.

80. The system of claim 79, wherein the tissue forms a part of an organ.

81. The system of claim 79, wherein the tissue forms a part of an internal organ.

82. The system of claim 79, wherein the tissue forms a portion of a tumor.

83. The system of claim 79, wherein the tissue forms a portion of an internal tumor.

84. The system of claim 79, wherein the tissue is a pathological tissue.

85. The system of claim 79, wherein the tissue forms a part of, or is associated with, a blood vessel tissue.

86. The system of claim 85, wherein said blood vessel tissue is selected from the group consisting of a blood clot, an occlusive plaque and a vulnerable plaque.

87. The system of claim 79, wherein the tissue forms a portion of a bone.

88. The system of claim 79, wherein the tissue is a stenotic tissue.

89. The method of claim 1, wherein said control unit is operable to measure an amplitude as a function of said frequency.

90. The method of claim 1, wherein said control unit is operable to measure a phase angle as a function of said frequency.

91. The system of claim 1, wherein said control unit is operable to calculate at least one mechanical property of the tissue from said frequency response spectrum.

92. The system of claim 91, wherein said mechanical property is an elastic constant.

93. The system of claim 91, wherein said mechanical property is selected from the group consisting of an elastic modulus, a Poisson's ratio, a shear modulus, a bulk modulus and a first Lamé coefficient.

94. The system of claim 79, wherein said position is on a skin of the body.

95. The system of claim 79, wherein said position is close to a blood vessel-of-interest.

96. The system of claim 95, wherein said blood vessel-of-interest is selected from the group consisting of a carotid, a femoral vessel and an abdominal aorta.

97. The system of claim 79, wherein said position is close to a lesion selected from the group consisting of a dermal lesion, a sub-dermal lesion and an internal lesion.

98. The system of claim 79, wherein said position is close to a bone.

99. The system of claim 79, wherein said position is close to a thorax.

100. The system of claim 79, wherein said mechanical vibrations generating assembly is operable to generate mechanical vibrations which are perpendicular to the body.

101. The system of claim 79, wherein said mechanical vibrations generating assembly is operable to generate mechanical vibrations which are inclined to the body by a predetermined inclination angle.

102. The system of claim 26, wherein said predetermined inclination angle is selected so as to enhance data acquisition.

103. The system of claim 79, wherein said frequency of said mechanical vibrations is selected from the group consisting of a single frequency, a superposition of a plurality of frequencies, a continuous frequency scan (chirp), and a band-limited white noise frequency.

104. The system of claim 79, wherein said mechanical vibrations generating assembly is constructed and designed so as to minimize effects of environmental noise.

105. The system of claim. 79, wherein said mechanical vibrations generating assembly comprises a mechanical linkage device for transferring said mechanical vibrations to the body.

106. The system of claim 105, wherein at least one of a size and a natural frequency of said mechanical linkage device is selected so as to increase dynamical interactions between the tissue and said at least one mechanical linkage device.

107. The system of claim 105, wherein said mechanical linkage device is characterized by a plurality of natural frequencies, and further wherein at least one frequency of said plurality of natural frequencies is higher than said frequency of said mechanical vibrations.

108. The system of claim 105, wherein said mechanical linkage device comprises a variable width beam spring.

109. The system of claim 105, wherein said mechanical linkage device comprises a strain gage for measuring displacement of said plurality of mechanical linkage devices.

110. The system of claim 105, wherein said mechanical linkage device comprises a proximity sensor for measuring displacement of said plurality of mechanical linkage devices.

111. The system of claim 79, further comprising at least one additional mechanical vibrations generating assembly having a plurality of mechanical linkage devices being in mutual communication, and operable to generate mechanical vibrations at a position adjacent to the predetermined location

112. The system of claim 111, wherein said plurality of mechanical linkage devices comprises a first mechanical linkage device and a second mechanical linkage device.

113. The system of claim 112, wherein said first and said second mechanical linkage devices are connected by at least one mechanical sensor, capable of receiving mechanical vibration therebetween.

114. The system of claim 112, wherein said first and said second mechanical linkage devices are connected by at least one connection rod.

115. The system of claim 112, wherein said first and said second mechanical linkage devices are each independently membranes.

116. The system of claim 115, wherein said membranes are made of a material selected from the group consisting of a plastic and a metal.

117. The system of claim 115, wherein said membranes are piezo-polymeric membranes.

118. The system of claim 79, wherein said mechanical vibrations generating assembly comprises at least one contact-tip.

119. The system of claim 118, wherein said at least one contact-tip comprises a plurality of contact-tips arranged in a matrix-like arrangement.

120. The system of claim 118, wherein said at least one contact-tip is sterilizable.

121. The system of claim 118, wherein said at least one contact-tip comprises at least one sterilizable cover.

122. The system of claim 118, wherein said at least one contact-tip is disposable.

123. The system of claim 79, wherein said mechanical vibrations generating assembly comprises a mechanical vibrations generating transducer assembly, said mechanical vibrations generating transducer assembly is operable to convert electrical signals into mechanical motions.

124. The system of claim 123, wherein said mechanical vibrations generating transducer assembly comprises a tubular transducer.

125. The system of claim 111, wherein said mechanical vibrations generating transducer assembly comprises a tubular transducer.

126. The system of claim 125, wherein said plurality of mechanical linkage devices comprises a first mechanical linkage device connected to a first end of said tubular transducer and a second mechanical linkage device connected to a second end of said tubular transducer.

127. The system of claim 123, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly and an electrostatic mechanical vibrations generating transducer assembly.

128. The system of claim 79, wherein said mechanical vibrations generating assembly comprises at least one mechanical sensor.

129. The system of claim 128, wherein said at least one mechanical sensor is selected from the group consisting of a contact sensor and a remote sensor.

130. The system of claim 128, wherein said at least one mechanical sensor is selected from the group consisting of an acceleration sensor, a force sensor, a pressure sensor and a displacement sensor.

131. The system of claim 79, wherein said mechanical vibrations generating assembly comprises a mechanism for isolating said mechanical vibrations generating assembly from environmental vibrations.

132. The system of claim 131, wherein said mechanism is operable to independently move in three orthogonal directions.

133. The system of claim 131, wherein said mechanism is operable to independently rotate in at least two orthogonal directions.

134. The system of claim 79, wherein said mechanical vibrations generating assembly is sizewise compatible with an anatomical system selected from the group consisting of the vascular system, the cardio-vascular system and the urinary system.

135. The system of claim 79, further comprising an imaging device for imaging the tissue.

136. The system of claim 135, wherein said imaging device is selected from the group consisting of an intra vascular ultra sound device, an intra vascular magnetic resonance device, a camera, a computer tomography device, and a magnetic resonance device.

137. The system of claim 135, wherein said imaging device is in communication with said control unit.

138. The system of claim 136, wherein said communication is selected from the group consisting of optical communication, electrical communication and acoustical communication.

139. The system of claim 135, wherein said imaging device is connected to said mechanical vibrations generating assembly.

140. The system of claim 134, wherein said mechanical vibrations generating assembly comprises at least one contact-tip.

141. The system of claim 140, wherein said mechanical vibrations generating assembly comprises a posing mechanism for bulging said at least one contact-tip out of an encapsulation of said mechanical vibrations generating assembly so as to touch the tissue.

142. The system of claim 141, wherein said mechanical vibrations generating assembly comprises at least one mechanical sensor.

143. The system of claim 142, wherein said mechanical vibrations generating assembly comprises a preamplifier, for at least partially amplifying electrical signals received from said at least one mechanical sensor.

144. The system of claim 79, wherein said control unit comprises a transmission unit for transmitting an electrical signal to said mechanical vibrations generating assembly.

145. The system of claim 133, wherein said transmission unit comprises a computerized synthesizer for generating a synthesized electrical pulse.

146. The system of claim 145, wherein said transmission unit further comprises a power amplifier for amplifying said synthesized electrical pulse.

147. The system of claim 79, wherein said control unit comprises a receiver for receiving an electrical signal from said mechanical vibrations generating assembly.

148. The system of claim 147, wherein said receiver comprises a preamplifier and a line amplifier, said preamplifier and said line amplifier configured and designed to amplify said electrical signal transmitted from said mechanical vibrations generating assembly.

149. The system of claim 148, wherein said receiver further comprises a display for displaying said electrical signal transmitted from said mechanical vibrations generating assembly.

150. The system of claim 149, wherein said display is selected from the group consisting of an oscilloscope, a spectrum analyzer, a processor display and a printer.

151. An endoscopic device for in vivo characterization of a tissue of a subject, the device comprising:
at least one imaging device for imaging the subject so as to determine a position of the tissue; and
at least one mechanical vibrations generating assembly for generating mechanical vibrations at said position of the tissue, and for measuring a frequency response spectrum of the tissue, said mechanical vibrations are at a frequency ranging from 10 Hz to 10 kHz.

152. The device of claim 151, wherein the tissue forms a part of, or is associated with, a blood vessel tissue.

153. The device of claim 152, wherein said blood vessel tissue is selected from the group consisting of a blood clot, an occlusive plaque and a vulnerable plaque.

154. The device of claim 151, wherein the tissue forms a part of, or is associated with, the urinary system of the subject.

155. The device of claim 151, wherein said mechanical vibrations generating assembly is operable to generate mechanical vibrations which are perpendicular to the tissue.

156. The device of claim 151, wherein said mechanical vibrations generating assembly is operable to generate mechanical vibrations which are inclined to the tissue by a predetermined inclination angle.

157. The device of claim 156, wherein said predetermined inclination angle is selected so as to enhance data acquisition.

158. The device of claim 151, wherein said frequency of said mechanical vibrations is selected from the group consisting of a single frequency, a superposition of a plurality of frequencies, a continuous frequency scan (chirp), and a band-limited white noise frequency.

159. The device of claim 151, wherein said mechanical vibrations generating assembly is constructed and designed so as to minimize effects of environmental noise.

160. The device of claim 151, wherein said mechanical vibrations generating assembly comprises at least one mechanical linkage device for transferring said mechanical vibrations to the tissue.

161. The device of claim 160, wherein at least one of a size and a natural frequency of said at least one mechanical linkage device is selected so as to increase dynamical interactions between the tissue and said at least one mechanical linkage device.

162. The device of claim 160, wherein said at least one mechanical linkage device is characterized by a plurality of natural frequencies, and further wherein at least one frequency of said plurality of natural frequencies is higher than said frequency of said mechanical vibrations.

163. The device of claim 160, wherein said at least one mechanical linkage device comprises a variable width beam spring.

164. The device of claim 160, wherein said at least one mechanical linkage device comprises a strain gage for measuring displacement of said at least one mechanical linkage device.

165. The device of claim 160, wherein said at least one mechanical linkage device comprises a proximity sensor for measuring displacement of said at least one mechanical linkage device.

166. The device of claim 160, wherein said at least one mechanical linkage device comprises a first mechanical linkage device and a second mechanical linkage device.

167. The device of claim 166, wherein said first and said second mechanical linkage devices are each independently membranes.

168. The device of claim 167, wherein said membranes are made of a material selected from the group consisting of a plastic and a metal.

169. The device of claim 167, wherein said membranes are piezo-polymeric membranes.

170. The device of claim 151, wherein said mechanical vibrations generating assembly comprises at least one contact-tip.

171. The device of claim 170, wherein said at least one contact-tip comprises a plurality of contact-tips arranged in a matrix-like arrangement.

172. The device of claim 151, wherein said mechanical vibrations generating assembly comprises a mechanical vibrations generating transducer assembly, said mechanical vibrations generating transducer assembly is operable to convert electrical signals into mechanical motions.

173. The device of claim 172, wherein said mechanical vibrations generating transducer assembly comprises a tubular transducer.

174. The device of claim 173, further comprising a first mechanical linkage device connected to a first end of said tubular transducer and a second mechanical linkage device connected to a second end of said tubular transducer.

175. The device of claim 174, wherein said first and said second mechanical linkage devices are connected by at least one mechanical sensor, capable of receiving mechanical vibration therebetween.

176. The device of claim 174, wherein said first and said second mechanical linkage devices are connected by at least one connection rod.

177. The device of claim 172, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical device (MEMS) vibrating generating transducer assembly and an electrostatic mechanical vibrations generating transducer assembly.

178. The device of claim 151, wherein said mechanical vibrations generating assembly comprises at least one mechanical sensor.

179. The device of claim 178, wherein said at least one mechanical sensor is selected from the group consisting of a contact sensor and a remote sensor.

180. The device of claim 178, wherein said at least one mechanical sensor is selected from the group consisting of an acceleration sensor, a force sensor, a pressure sensor and a displacement sensor.

181. The device of claim 200, wherein said imaging device is selected from the group consisting of an intra vascular ultra sound device, an intra vascular magnetic resonance device and a camera.

182. The device of claim 151, wherein said mechanical vibrations generating assembly comprises at least one contact-tip.

183. The device of claim 182, wherein said mechanical vibrations generating assembly comprises a posing mechanism for bulging said at least one contact-tip out of an encapsulation of said mechanical vibrations generating assembly so as to touch the tissue.

184. The device of claim 183, wherein said mechanical vibrations generating assembly comprises at least one mechanical sensor.

185. The device of claim 184, wherein said mechanical vibrations generating assembly comprises a preamplifier, for at least partially amplifying electrical signals received from said at least one mechanical sensor.

186. A system for in vivo characterization of a tissue of a subject, the system comprising:
an endoscopic device having at least one imaging device and at least one mechanical vibrations generating assembly, said at least one imaging device being for imaging the subject and said at least one mechanical vibrations generating assembly being for generating mechanical vibrations at a position of the tissue, said mechanical vibrations are at a frequency ranging from 10 Hz to 10 kHz; and
a control unit for scanning said frequency of said mechanical vibrations, and for measuring a frequency response spectrum from the tissue, thereby to characterize the tissue.

187. The system of claim 186, wherein the tissue forms a part of, or is associated with, a blood vessel tissue.

188. The system of claim 187, wherein said blood vessel tissue is selected from the group consisting of a blood clot, an occlusive plaque and a vulnerable plaque.

189. The system of claim 186, wherein the tissue forms a part of, or is associated with, the urinary system of the subject.

190. The system of claim 186, wherein said control unit is operable to measure an amplitude as a function of said frequency.

191. The system of claim 186, wherein said control unit is operable to measure a phase angle as a function of said frequency.

192. The system of claim 186, wherein said control unit is operable to calculate at least one mechanical property of the tissue from said frequency response spectrum.

193. The system of claim 192, wherein said mechanical property is an elastic constant.

194. The system of claim 192, wherein said mechanical property is selected from the group consisting of an elastic modulus, a Poisson's ratio, a shear modulus, a bulk modulus and a first Lamé coefficient.

195. The system of claim 186, wherein said position is close to a blood vessel-of-interest.

196. The system of claim 195, wherein said blood vessel-of-interest is selected from the group consisting of a carotid, a femoral vessel and an abdominal aorta.

197. The system of claim 186, wherein said position is close to an internal lesion.

198. The system of claim 186, wherein said mechanical vibrations generating assembly is operable to generate mechanical vibrations which are perpendicular to the tissue.

199. The system of claim 186, wherein said mechanical vibrations generating assembly is operable to generate mechanical vibrations which are inclined to the tissue by a predetermined inclination angle.

200. The system of claim 199, wherein said predetermined inclination angle is selected so as to enhance data acquisition.

201. The system of claim 186, wherein said frequency of said mechanical vibrations is selected from the group consisting of a single frequency, a superposition of a plurality of frequencies, a continuous frequency scan (chirp), and a band-limited white noise frequency.

202. The system of claim 186, wherein said mechanical vibrations generating assembly comprises at least one mechanical linkage device for transferring said mechanical vibrations to the tissue.

203. The system of claim 202, wherein at least one of a size and a natural frequency of said at least one mechanical linkage device is selected so as to increase dynamical interactions between the tissue and said at least one mechanical linkage device.

204. The system of claim 202, wherein said at least one mechanical linkage device is characterized by a plurality of natural frequencies, and further wherein at least one frequency of said plurality of natural frequencies is higher than said frequency of said mechanical vibrations.

205. The system of claim 202, wherein said at least one mechanical linkage device comprises a variable width beam spring.

206. The system of claim 202, wherein said at least one mechanical linkage device comprises a strain gage for measuring displacement of said at least one mechanical linkage device.

207. The system of claim 202, wherein said at least one mechanical linkage device comprises a proximity sensor for measuring displacement of said at least one mechanical linkage device.

208. The system of claim 202, wherein said at least one mechanical linkage device comprises a first mechanical linkage device and a second mechanical linkage device.

209. The system of claim 208, wherein said first and said second mechanical linkage devices are each independently membranes.

210. The system of claim 209, wherein said membranes are made of a material selected from the group consisting of a plastic and a metal.

211. The system of claim 209, wherein said membranes are piezo-polymeric membranes.

212. The system of claim 186, wherein said mechanical vibrations generating assembly comprises at least one contact-tip.

213. The system of claim 212, wherein said at least one contact-tip comprises a plurality of contact-tips arranged in a matrix-like arrangement.

214. The system of claim 186, wherein said mechanical vibrations generating assembly comprises a mechanical vibrations generating transducer assembly, said mechanical vibrations generating transducer assembly is operable to convert electrical signals into mechanical motions.

215. The system of claim 214, wherein said mechanical vibrations generating transducer assembly comprises a tubular transducer.

216. The system of claim 215, further comprising a first mechanical linkage device connected to a first end of said tubular transducer and a second mechanical linkage device connected to a second end of said tubular transducer.

217. The system of claim 216, wherein said first and said second mechanical linkage devices are connected by at least one mechanical sensor, capable of receiving mechanical vibration therebetween.

218. The system of claim 214, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly and an electrostatic mechanical vibrations generating transducer assembly.

219. The system of claim 186, wherein said mechanical vibrations generating assembly comprises at least one mechanical sensor.

220. The system of claim 219, wherein said at least one mechanical sensor is selected from the group consisting of a contact sensor and a remote sensor.

221. The system of claim 219, wherein said at least one mechanical sensor is selected from the group consisting of an acceleration sensor, a force sensor, a pressure sensor and a displacement sensor.

222. The system of claim 186, wherein said imaging device is selected from the group consisting of an intra vascular ultra sound device, an intra vascular magnetic resonance device and a camera.

223. The system of claim 186, wherein said mechanical vibrations generating assembly comprises at least one contact-tip.

224. The system of claim 223, wherein said mechanical vibrations generating assembly comprises a posing mechanism for bulging said at least one contact-tip out of an encapsulation of said mechanical vibrations generating assembly so as to touch the tissue.

225. The system of claim 224, wherein said mechanical vibrations generating assembly comprises at least one mechanical sensor.

226. The system of claim 225, wherein said mechanical vibrations generating assembly comprises a preamplifier, for at least partially amplifying electrical signals received from said at least one mechanical sensor.

227. The system of claim 186, wherein said control unit comprises a transmission unit for transmitting an electrical signal to said mechanical vibrations generating assembly.

228. The system of claim 227, wherein said transmission unit comprises a computerized synthesizer for generating a synthesized electrical pulse.

229. The system of claim 228, wherein said transmission unit further comprises a power amplifier for amplifying said synthesized electrical pulse.

230. The system of claim 186, wherein said control unit comprises a receiver for receiving an electrical signal from said mechanical vibrations generating assembly.

231. The system of claim 230, wherein said receiver comprises a preamplifier and a line amplifier, said preamplifier and said line amplifier configured and designed to amplify said electrical signal transmitted from said mechanical vibrations generating assembly.

232. The system of claim 231, wherein said receiver further comprises a display for displaying said electrical signal transmitted from said mechanical vibrations generating assembly.

233. The system of claim 232, wherein said display is selected from the group consisting of an oscilloscope, a spectrum analyzer, a processor display and a printer.

234. A method of characterizing a tissue of a subject, the method comprising:
(a) endoscopically inserting an endoscopic device into the subject, and using said endoscopic device for (i) imaging the subject so as to determine a position of the tissue; and (ii) generating mechanical vibrations at said position, said mechanical vibrations being at a frequency ranging from 10 Hz to 10 kHz;
(b) scanning said frequency of said mechanical vibrations; and
(c) measuring a frequency response spectrum from the tissue;
thereby characterizing the tissue.

235. The method of claim 234, wherein the tissue forms a part of, or is associated with, a blood vessel tissue.

236. The method of claim 235, wherein said blood vessel tissue is selected from the group consisting of a blood clot, an occlusive plaque and a vulnerable plaque.

237. The method of claim 234, wherein the tissue forms a part of, or is associated with, the urinary system of the subject.

238. The method of claim 234, further comprising measuring an amplitude as a function of said frequency.

239. The method of claim 234, further comprising measuring a phase angle as a function of said frequency.

240. The method of claim 234, further comprising calculating at least one mechanical property of the tissue from said frequency response spectrum.

241. The method of claim 240, wherein said mechanical property is an elastic constant.

242. The method of claim 240, wherein said mechanical property is selected from the group consisting of an elastic modulus, a Poisson's ratio, a shear modulus, a bulk modulus and a first Lamé coefficient.

243. The method of claim 234, wherein said position is close to a blood vessel-of-interest.

244. The method of claim 243, wherein said blood vessel-of-interest is selected from the group consisting of a carotid, a femoral vessel and an abdominal aorta.

245. The method of claim 234, wherein said position is close to an internal lesion.

246. The method of claim 234, wherein said mechanical vibrations are perpendicular to the tissue.

247. The method of claim 234, wherein said mechanical vibrations are inclined to the tissue by a predetermined inclination angle.

248. The method of claim 247, wherein said predetermined inclination angle is selected so as to enhance data acquisition.

249. The method of claim 234, wherein said frequency of said mechanical vibrations is selected from the group consisting of a single frequency, a superposition of a plurality of frequencies, a continuous frequency scan (chirp), and a band-limited white noise frequency.

250. The method of claim 234, wherein said generating said mechanical vibrations is by a mechanical vibrations generating assembly.

251. The method of claim 234, wherein said mechanical vibrations generating assembly comprises at least one mechanical linkage device for transferring said mechanical vibrations to the tissue.

252. The method of claim 251, wherein at least one of a size and a natural frequency of said at least one mechanical linkage device is selected so as to increase dynamical interactions between the tissue and said at least one mechanical linkage device.

253. The method of claim 251, wherein said at least one mechanical linkage device is characterized by a plurality of natural frequencies, and further wherein at least one frequency of said plurality of natural frequencies is higher than said frequency of said mechanical vibrations.

254. The method of claim 251, wherein said at least one mechanical linkage device comprises a variable width beam spring.

255. The method of claim 251, wherein said at least one mechanical linkage device comprises a strain gage for measuring displacement of said at least one mechanical linkage device.

256. The method of claim 251, wherein said at least one mechanical linkage device comprises a proximity sensor for measuring displacement of said at least one mechanical linkage device.

257. The method of claim 234, wherein said generating said mechanical vibrations is by transmitting mechanical vibration from a first mechanical linkage device to a second mechanical linkage device via at least one mechanical sensor.

258. The method of claim 257, wherein said first and said second mechanical linkage devices are each independently membranes.

259. The method of claim 258, wherein said membranes are made of a material selected from the group consisting of a plastic and a metal.

260. The method of claim 258, wherein said membranes are piezo-polymeric membranes.

261. The method of claim 234, further comprising converting electrical signals into mechanical motions using a mechanical vibrations generating transducer assembly.

262. The method of claim 261, wherein said mechanical vibrations generating transducer assembly comprises a tubular transducer.

263. The method of claim 250, wherein said mechanical vibrations generating assembly comprises at least one mechanical sensor.

264. The method of claim 263, wherein said at least one mechanical sensor is selected from the group consisting of a contact sensor and a remote sensor.

265. The method of claim 263, wherein said at least one mechanical sensor is selected from the group consisting of an acceleration sensor, a force sensor, a pressure sensor and a displacement sensor.

266. The method of claim 234, wherein said endoscopic device comprises an imaging device, selected from the group consisting of an intra vascular ultra sound device, an intra vascular magnetic resonance device and a camera.

267. The method of claim 250, wherein said mechanical vibrations generating assembly comprises at least one contact-tip.

268. The method of claim 267, further comprising bulging said at least one contact-tip out of an encapsulation of said mechanical vibrations generating assembly so as to touch the tissue.

269. The method of claim 268, wherein said mechanical vibrations generating assembly comprises at least one mechanical sensor.

270. The method of claim 269, further comprising at least partially amplifying electrical signals received from said at least one mechanical sensor.

271. The method of claim 250, further comprising transmitting an electrical signal to said mechanical vibrations generating assembly.

272. The method of claim 271, wherein said transmitting said electrical comprises generating a synthesized electrical pulse.

273. The method of claim 272, further comprising amplifying said synthesized electrical pulse.

274. The method of claim 250, further comprising amplifying electrical signal transmitted from said mechanical vibrations generating assembly.

275. The method of claim 274, further comprising displaying said electrical signal transmitted from said mechanical vibrations generating assembly.

276. The method of claim 275, wherein said display is selected from the group consisting of an oscilloscope, a spectrum analyzer, a processor display and a printer.

277. The method of claim 234, further comprising classifying said frequency response spectrum.

278. The method of claim 277, wherein said classifying said frequency response spectrum comprises:
(a) identifying resonance peak maxima of said frequency response spectrum;
(b) from said resonance peak maxima, determining a first type of maximum being indicative of a first type of tissue, and a second type of maximum being indicative of a second type of tissue.
(c) using said first type of maximum and said second type of maximum to classify said first and said types of tissue.

279. The method of claim 278, wherein said step (c) comprises calculating a ratio between said first type of maximum and said second type of maximum.

280. The method of claim 278, further comprising averaging said resonance peak maxima.

281. The method of claim 278, wherein said first and said second types of maxima are determined by absolute values of said resonance peak maxima.

282. The method of claim 278, wherein said first and said second types of maxima are determined by shapes of said resonance peak maxima.

283. The method of claim 278, wherein said first and said second types of maxima are determined by frequency shifts of said resonance peak maxima.

284. A system for characterizing a tissue present in a predetermined location of a body of a subject, the system comprising:
at least one mechanical vibrations generating assembly each having a plurality of mechanical linkage devices being in mutual communication, and operable to generate mechanical vibrations at a position adjacent to the predetermined location, said mechanical vibrations being at a frequency ranging from 10 Hz to 10 kHz; and
a control unit for scanning said frequency of said mechanical vibrations, and for measuring a frequency response spectrum from the tissue, thereby to characterize the tissue.

285. The system of claim 284, wherein the tissue forms a part of an organ.

286. The system of claim 284, wherein the tissue forms a part of an internal organ.

287. The system of claim 284, wherein the tissue forms a portion of a tumor.

288. The system of claim 284, wherein the tissue forms a portion of an internal tumor.

289. The system of claim 284, wherein the tissue is a pathological tissue.

290. The system of claim 284, wherein the tissue forms a part of, or is associated with, a blood vessel tissue.

291. The system of claim 290, wherein said blood vessel tissue is selected from the group consisting of a blood clot, an occlusive plaque and a vulnerable plaque.

292. The system of claim 284, wherein the tissue forms a portion of a bone.

293. The system of claim 284, wherein the tissue is a stenotic tissue.

294. The system of claim 284, wherein at least one of a size and a natural frequency of said plurality of mechanical linkage devices is selected so as to increase dynamical interactions between the tissue and said at least one mechanical linkage device.

295. The system of claim 284, wherein said at least one mechanical linkage device is characterized by a plurality of natural frequencies, and further wherein at least one frequency of said plurality of natural frequencies is higher than said frequency of said mechanical vibrations.

296. The system of claim 284, wherein said plurality of mechanical linkage devices comprises a variable width beam spring.

297. The system of claim 284, wherein said plurality of mechanical linkage devices comprises a strain gage for measuring displacement of said plurality of mechanical linkage devices.

298. The system of claim 284, wherein said plurality of mechanical linkage devices comprises a proximity sensor for measuring displacement of said plurality of mechanical linkage devices.

299. The system of claim 284, wherein said control unit is operable to measure an amplitude as a function of said frequency.

300. The system of claim 284, wherein said control unit is operable to measure a phase angle as a function of said frequency.

301. The system of claim 284, wherein said control unit is operable to calculate at least one mechanical property of the tissue from said frequency response spectrum.

302. The system of claim 301, wherein said mechanical property is an elastic constant.

303. The system of claim 301, wherein said mechanical property is selected from the group consisting of an elastic modulus, a Poisson's ratio, a shear modulus, a bulk modulus and a first Lamé coefficient.

304. The system of claim 284, wherein said position is on a skin of the body.

305. The system of claim 284, wherein said position is close to a blood vessel-of-interest.

306. The system of claim 305, wherein said blood vessel-of-interest is selected from the group consisting of a carotid, a femoral vessel and an abdominal aorta.

307. The system of claim 284, wherein said position is close to a lesion selected from the group consisting of a dermal lesion, a sub-dermal lesion and an internal lesion.

308. The system of claim 284, wherein said position is close to a bone.

309. The system of claim 284, wherein said position is close to a thorax.

310. The system of claim 284, wherein said mechanical vibrations generating assembly is operable to generate mechanical vibrations which are perpendicular to the body.

311. The system of claim 284, wherein said mechanical vibrations generating assembly is operable to generate mechanical vibrations which are inclined to the body by a predetermined inclination angle.

312. The system of claim 311, wherein said predetermined inclination angle is selected so as to enhance data acquisition.

313. The system of claim 284, wherein said frequency of said mechanical vibrations is selected from the group consisting of a single frequency, a superposition of a plurality of frequencies, a continuous frequency scan (chirp), and a band-limited white noise frequency.

314. The system of claim 284, wherein said mechanical vibrations generating assembly is constructed and designed so as to minimize effects of environmental noise.

315. The system of claim 284, wherein said plurality of mechanical linkage devices comprises a first mechanical linkage device and a second mechanical linkage device.

316. The system of claim 315, wherein said first and said second mechanical linkage devices are connected by at least one mechanical sensor, capable of receiving mechanical vibration therebetween.

317. The system of claim 315, wherein said first and said second mechanical linkage devices are connected by at least one connection rod.

318. The system of claim 315, wherein said first and said second mechanical linkage devices are each independently membranes.

319. The system of claim 318, wherein said membranes are made of a material selected from the group consisting of a plastic and a metal.

320. The system of claim 318, wherein said membranes are piezo-polymeric membranes.

321. The system of claim 284, wherein said mechanical vibrations generating assembly comprises at least one contact-tip.

322. The system of claim 321, wherein said at least one contact-tip comprises a plurality of contact-tips arranged in a matrix-like arrangement.

323. The system of claim 321, wherein said at least one contact-tip is sterilizable.

324. The system of claim 321, wherein said at least one contact-tip comprises at least one sterilizable cover.

325. The system of claim 321, wherein said at least one contact-tip is disposable.

326. The system of claim 284, wherein said mechanical vibrations generating assembly comprises a mechanical vibrations generating transducer assembly, said mechanical vibrations generating transducer assembly is operable to convert electrical signals into mechanical motions.

327. The system of claim 326, wherein said mechanical vibrations generating transducer assembly comprises a tubular transducer.

328. The system of claim 327, wherein said plurality of mechanical linkage devices comprises a first mechanical linkage device connected to a first end of said tubular transducer and a second mechanical linkage device connected to a second end of said tubular transducer.

329. The system of claim 326, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly and an electrostatic mechanical vibrations generating transducer assembly.

330. The system of claim 284, wherein said at least one mechanical sensor is selected from the group consisting of a contact sensor and a remote sensor.

331. The system of claim 284, wherein said at least one mechanical sensor is selected from the group consisting of an acceleration sensor, a force sensor, a pressure sensor and a displacement sensor.

332. The system of claim 284, wherein said mechanical vibrations generating assembly comprises a mechanism for isolating said mechanical vibrations generating assembly from environmental vibrations.

333. The system of claim 332, wherein said mechanism is operable to independently move in three orthogonal directions.

334. The system of claim 332, wherein said mechanism is operable to independently rotate in at least two orthogonal directions.

335. The system of claim 284, wherein said mechanical vibrations generating assembly is sizewise compatible with an anatomical system selected from the group consisting of the vascular system, the cardio-vascular system and the urinary system.

336. The system of claim 284, further comprising an imaging device for imaging the tissue.

337. The system of claim 336, wherein said imaging device is selected from the group consisting of an intra vascular ultra sound device, an intra vascular magnetic resonance device, a camera, a computer tomography device, and a magnetic resonance device.

338. The system of claim 336, wherein said imaging device is in communication with said control unit.

339. The system of claim 338, wherein said communication is selected from the group consisting of optical communication, electrical communication and acoustical communication.

340. The system of claim 336, wherein said imaging device is connected to said mechanical vibrations generating assembly.

341. The system of claim 335, wherein said mechanical vibrations generating assembly comprises at least one contact-tip.

342. The system of claim 341, wherein said mechanical vibrations generating assembly comprises a posing mechanism for bulging said at least one contact-tip out of an encapsulation of said mechanical vibrations generating assembly so as to touch the tissue.

343. The system of claim 342, wherein said mechanical vibrations generating assembly comprises at least one mechanical sensor.

344. The system of claim 343, wherein said mechanical vibrations generating assembly comprises a preamplifier, for at least partially amplifying electrical signals received from said at least one mechanical sensor.

345. The system of claim 284, wherein said control unit comprises a transmission unit for transmitting an electrical signal to said mechanical vibrations generating assembly.

346. The system of claim 334, wherein said transmission unit comprises a computerized synthesizer for generating a synthesized electrical pulse.

347. The system of claim 346, wherein said transmission unit further comprises a power amplifier for amplifying said synthesized electrical pulse.

348. The system of claim 284, wherein said control unit comprises a receiver for receiving an electrical signal from said mechanical vibrations generating assembly.

349. The system of claim 348, wherein said receiver comprises a preamplifier and a line amplifier, said preamplifier and said line amplifier configured and designed to amplify said electrical signal transmitted from said mechanical vibrations generating assembly.

350. The system of claim 349, wherein said receiver further comprises a display for displaying said electrical signal transmitted from said mechanical vibrations generating assembly.

351. The system of claim 350, wherein said display is selected from the group consisting of an oscilloscope, a spectrum analyzer, a processor display and a printer.

352. A mechanical vibrations generating assembly for generating mechanical vibrations at a position of a body of a subject, comprising a transducer assembly, a first mechanical linkage device, connected to a first end of said transducer assembly, and a second mechanical linkage device, connected to a second end of said transducer assembly;
wherein said transducer assembly, said first mechanical linkage device and said second mechanical linkage device are constructed and designed so that when electrical signals are inputted to said transducer assembly, said electrical signals are converted into mechanical motions, and said first and said second mechanical linkage devices generates the mechanical vibrations.

353. The mechanical vibrations generating assembly of claim 352, further comprising at least one additional mechanical linkage device, mechanically communicating with said transducer assembly.

354. The mechanical vibrations generating assembly of claim 352, wherein said first and said second mechanical linkage devices are each independently membranes.

355. The mechanical vibrations generating assembly of claim 352, wherein said membranes are made of a material selected from the group consisting of a plastic and a metal.

356. The mechanical vibrations generating assembly of claim 352, wherein said membranes are piezo-polymeric membranes.

357. The mechanical vibrations generating assembly of claim 352, further comprising at least one contact-tip, connected to at least one of said mechanical linkage devices.

358. The mechanical vibrations generating assembly of claim 352, wherein said at least one contact-tip comprises a plurality of contact-tips arranged in a matrix-like arrangement.

359. The mechanical vibrations generating assembly of claim 352, wherein said at least one contact-tip is sterilizable.

360. The mechanical vibrations generating assembly of claim 352, wherein said at least one contact-tip comprises at least one sterilizable cover.

361. The mechanical vibrations generating assembly of claim 352, wherein said at least one contact-tip is disposable.

362. The mechanical vibrations generating assembly of claim 352, wherein at least one of a size and a natural frequency of said mechanical linkage devices is selected so as to increase dynamical interactions between the a portion of the body and said mechanical linkage devices.

363. The mechanical vibrations generating assembly of claim 352, wherein said mechanical linkage devices are characterized by a plurality of natural frequencies, and further wherein at least one frequency of said plurality of natural frequencies is higher than a frequency of the mechanical vibrations.

364. The mechanical vibrations generating assembly of claim 352, wherein said plurality of mechanical linkage devices comprises a strain gage for measuring displacement of said mechanical linkage devices.

365. The mechanical vibrations generating assembly of claim 352, wherein said mechanical linkage devices comprises a proximity sensor for measuring displacement of said mechanical linkage devices.

366. The mechanical vibrations generating assembly of claim 352, wherein said transducer assembly comprises a tubular transducer.

367. The mechanical vibrations generating assembly of claim 352, further comprising at least one mechanical sensor.

368. The mechanical vibrations generating assembly of claim 352, further comprising at least one mechanical sensor connecting said first mechanical linkage device and said mechanical linkage device, said at least one mechanical sensor being capable of receiving mechanical vibration therethrough.

369. The mechanical vibrations generating assembly of claim 367, wherein said at least one mechanical sensor is selected from the group consisting of a contact sensor and a remote sensor.

370. The mechanical vibrations generating assembly of claim 367, wherein said at least one mechanical sensor is selected from the group consisting of an acceleration sensor, a force sensor, a pressure sensor and a displacement sensor.

371. A method of classifying a frequency response spectrum of a structural material, the method is executable by a data processor and comprising;
(a) constructing a physical model of a plurality of harmonic oscillators, said physical model comprises a set of parameters and being characterized by a plurality of equations of motion;
(b) simultaneously solving said plurality of equations of motion so as to provide at least one frequency response; and
(c) comparing said at least one frequency response with the frequency response spectrum of the structural material, thereby classifying the frequency response spectrum of the structural material.

372. The method of claim 371, wherein said physical model is an N degree-of-freedom physical model, said N is a positive integer.

373. The method of claim 371, wherein said plurality of harmonic oscillators are coupled harmonic oscillators.

374. The method of claim 371, wherein at least a portion of said plurality of harmonic oscillators are damped harmonic oscillators.

375. The method of claim 371, wherein at least a portion of said plurality of harmonic oscillators are forced harmonic oscillators.

376. The method of claim 371, wherein said set of parameters comprises at least one constant of inertia and at least one elastic constant.

377. The method of claim 376, wherein said constant of inertia is mass and further wherein said elastic constant is a spring constant.

378. The method of claim 376, wherein said constant of inertia is inductance and further wherein said elastic constant is a reciprocal of capacitance.

379. The method of claim 371, further comprising repeating said steps (a)-(c) at least once, each time using a different set of parameters.

380. The method of claim 371, wherein said set of parameters represent dynamic stiffness and density of the structural material.

381. An apparatus for classifying a frequency response spectrum of a structural material, the apparatus comprising;
(a) a constructor for constructing a physical model of a plurality of harmonic oscillators, said physical model comprises a set of parameters and being characterized by a plurality of equations of motion;
(b) a solver for simultaneously solving said plurality of equations of motion so as to provide at least one frequency response; and
(c) a comparing unit for comparing said at least one frequency response with the frequency response spectrum of the structural material, thereby to classify the frequency response spectrum of the structural material.

382. The apparatus of claim 381, wherein said physical model is an N degree-of-freedom physical model, said N is a positive integer.

383. The apparatus of claim 381, wherein said plurality of harmonic oscillators are coupled harmonic oscillators.

384. The apparatus of claim 381, wherein at least a portion of said plurality of harmonic oscillators are damped harmonic oscillators.

385. The apparatus of claim 381, wherein at least a portion of said plurality of harmonic oscillators are forced harmonic oscillators.

386. The apparatus of claim 381, wherein said set of parameters comprises at least one constant of inertia and at least one elastic constant.

387. The apparatus of claim 386, wherein said constant of inertia is mass and further wherein said elastic constant is a spring constant.

388. The apparatus of claim 386, wherein said constant of inertia is inductance and further wherein said elastic constant is a reciprocal of capacitance.

389. The apparatus of claim 381, wherein said set of parameters represent dynamic stiffness and density of the structural material.

390. A method of constructing a frequency resonance spectra library the frequency resonance spectra characterizing a plurality of tissues of a plurality of subjects, the method comprising, for each subject:
(a) selecting a tissue of said subject and generating mechanical vibrations at a position adjacent to said tissue, said mechanical vibrations are at a frequency ranging from 10 Hz to 10 kHz;
(b) scanning said frequency of said mechanical vibrations;
(c) measuring a frequency response spectrum from of said tissue; and
(d) recording said frequency response spectrum;
thereby providing a frequency response spectrum entry of the library, said frequency response spectrum entry characterizing said tissue, thereby constructing the frequency resonance spectra library.

391. The method of claim 390, wherein said tissue forms a part of an organ.

392. The method of claim 390, wherein said tissue forms a part of an internal organ.

393. The method of claim 390, wherein said tissue forms a portion of a tumor.

394. The method of claim 390, wherein said tissue forms a portion of an internal tumor.

395. The method of claim 390, wherein said tissue is a pathological tissue.

396. The method of claim 390, wherein said tissue forms a part of, or is associated with, a blood vessel tissue.

397. The method of claim 396, wherein said blood vessel tissue is selected from the group consisting of a blood clot, an occlusive plaque and a vulnerable plaque.

398. The method of claim 397, wherein said blood vessel is selected from the group consisting of a carotid, a femoral, and an abdominal aorta.

399. The method of claim 390, wherein said tissue forms a portion of a bone.

400. The method of claim 390, wherein said tissue is a stenotic tissue.

401. The method of claim 390, wherein said tissue is a lesion.

402. The method of claim 390, wherein said lesion is selected from the group consisting of a dermal lesion, a sub-dermal lesion and an internal lesion.

403. The method of claim 390, wherein said adjacent to said tissue is on a skin of said body.

404. The method of claim 390, wherein said mechanical vibrations are perpendicular to said body.

405. The method of claim 390, wherein said generating said mechanical vibrations is performed such that said mechanical vibrations are inclined to said body, by a predetermined inclination angle.

406. The method of claim 405, wherein said predetermined inclination angle is selected so as to enhance data acquisition.

407. The method of claim 405, wherein said step of generating mechanical vibrations is repeated a plurality of times, each time with a different inclination angle.

408. The method of claim 390, wherein said step of generating mechanical vibrations is repeated a plurality of times, each time for a different tissue.

409. The method of claim 390, wherein said frequency of said mechanical vibrations is selected from the group consisting of a single frequency, a superposition of a plurality of frequencies, a continuous frequency scan (chirp), and a band-limited white noise frequency.

410. The method of claim 390, wherein said generating said mechanical vibrations is by a mechanical vibrations generating assembly.

411. The method of claim 390, wherein said mechanical vibrations generating assembly is constructed and designed so as to minimize effects of environmental noise.

412. The method of claim 411, wherein said mechanical vibrations generating assembly comprises a mechanical linkage device for transferring said mechanical vibrations to said body.

413. The method of claim 390, wherein said mechanical vibrations generating assembly comprises at least one contact-tip.

414. The method of claim 413, wherein said at least one contact-tip comprises a plurality of contact-tips arranged in a matrix-like arrangement.

415. The method of claim 413, wherein said at least one contact-tip is sterilizable.

416. The method of claim 413, wherein said at least one contact-tip comprises at least one sterilizable cover.

417. The method of claim 413, wherein said at least one contact-tip is disposable.

418. The method of claim 410, wherein said mechanical vibrations generating assembly comprises a mechanical vibrations generating transducer assembly, said mechanical vibrations generating transducer assembly is operable to convert electrical signals into mechanical motions.

419. The method of claim 418, wherein said mechanical vibrations generating transducer assembly is selected from the group consisting of a piezoelectric mechanical vibrations generating transducer assembly, an electric mechanical vibrations generating transducer assembly, an electrostrictive mechanical vibrations generating transducer assembly, a magnetic mechanical vibrations generating transducer assembly, a magnetostrictive mechanical vibrations generating transducer assembly, an electromagnetic mechanical vibrations generating transducer assembly, a micro electro mechanical system (MEMS) vibrating generating transducer assembly, and an electrostatic mechanical vibrations generating transducer assembly.

420. The method of claim 410, wherein said mechanical vibrations generating assembly comprises at least one mechanical sensor.

421. The method of claim 420, wherein said at least one mechanical sensor is selected from the group consisting of a contact sensor and a remote sensor.

422. The method of claim 420, wherein said at least one mechanical sensor is selected from the group consisting of an acceleration sensor, a force sensor, a pressure sensor and a displacement sensor.

423. The method of claim 410, wherein said mechanical vibrations generating assembly comprises a mechanism for isolating said mechanical vibrations generating assembly from environmental vibrations.

424. The method of claim 423, wherein said mechanism is operable to independently move in three orthogonal directions.

425. The method of claim 423, wherein said mechanism is operable to independently rotate in at least two orthogonal directions.

426. The method of claim 410, further comprising transmitting an electrical signal to said mechanical vibrations generating assembly.

427. The method of claim 410, wherein said measuring is by receiving an electrical signal transmitted from said mechanical vibrations generating assembly.

428. The method of claim 426, further comprising displaying said electrical signal transmitted from said mechanical vibrations generating assembly on a display.

429. The method of claim 428, wherein said display is selected from the group consisting of an oscilloscope, a spectrum analyzer, a processor display and a printer.

430. A resonance spectra library produced by the method of claim 390, the resonance spectra of the library are stored, in a retrievable and/or displayable format, on a memory media.

431. A memory media, storing in a retrievable and/or displayable format the resonance spectra of the resonance spectra library of claim 430.
